(19) United States
(12) Patent Application Publication
(10) Pub. No.: US 2024/0407668 A1
(43) Pub. Date: Dec. 12, 2024

(54) GAIT MEASUREMENT DEVICE, MEASUREMENT DEVICE, GAIT MEASUREMENT SYSTEM, GAIT MEASUREMENT METHOD, AND RECORDING MEDIUM

(71) Applicant: NEC Corporation, Minato-ku, Tokyo (JP)

(72) Inventor: Hiroshi KAJITANI, Tokyo (JP)

(73) Assignee: NEC Corporation, Minato-ku, Tokyo (JP)

(21) Appl. No.: 18/703,784

(22) PCT Filed: Mar. 11, 2022
(86) PCT No.: PCT/JP2022/011003

§ 371 Date: Apr. 23, 2024

(51) Int. Cl. A61B 5/11; A61B 5/00

(52) U.S. Cl. CPC A61B 5/112; A61B 5/6807; A61B 2562/0219

(57) ABSTRACT

Provided is a gait measurement device that includes an acquisition unit that acquires sensor data measured by a sensor mounted on footwear, a mounting-direction-determination unit that determines a mounting direction of the sensor using the acquired sensor data, a coordinate conversion unit that converts a coordinate system of the sensor data in accordance with the determined mounting direction of the sensor, a detection unit that detects a walking event from time-series data of the sensor data in which the coordinate system has been converted, a calculation unit that calculates a gait parameter in accordance with the detected walking event, and a transmission unit that transmits the calculated gait parameter.

### TECHNICAL FIELD

The present disclosure relates to a gait measurement device and the like that measure a gait using sensor data measured by a sensor mounted on footwear.

### BACKGROUND ART

With increasing interest in healthcare, services for providing information according to features (also referred to as a gait) included in a walking pattern have attracted attention. For example, a technique for analyzing a gait based on sensor data measured by a measurement device mounted on footwear such as shoes has been developed. In such a measurement device, a sensor such as an acceleration sensor or an angular velocity sensor is mounted.

PTL 1 discloses a device that detects an abnormality of a foot based on the walking features of a pedestrian. The device of PTL 1 extracts a feature amount (also referred to as a walking feature amount) relevant to the walking of the pedestrian wearing footwear using data acquired by a sensor installed on the footwear. The device of PTL 1 detects an abnormality of the foot of the pedestrian based on the extracted walking feature amount.

### CITATION LIST

### Patent Literature

PTL 1: WO 2021/140658 A1

### SUMMARY OF INVENTION

### Technical Problem

In a method of PTL 1, the abnormality of the foot of the pedestrian is estimated using the walking feature amount extracted from the data acquired by the sensor installed on the footwear. The sensor installed on the footwear includes a sensor such as an acceleration sensor and an angular velocity sensor. In the measurement device of PTL 1, firmware optimized in a normal mounting direction of the sensor is implemented. Therefore, in response to a change in the mounting direction of the sensor, various threshold value determinations are changed, and the gait measurement is not available. In such a case, it is necessary to mount the sensor again so that the mounting direction of the sensor is normal. Mounting the sensor again after a user starts walking leads to the deterioration of usability. A situation that has not been measured after the user finishes walking also leads to the deterioration of the usability. Therefore, it is required to measure sensor data relevant to the motion of the foot regardless of the mounting direction of the sensor.

An object of the present disclosure is to provide a gait measurement device and the like capable of measuring sensor data relevant to the motion of a foot regardless of a mounting direction of a sensor.

### Solution to Problem

A gait measurement device of one aspect of the present disclosure includes an acquisition unit that acquires sensor data measured by a sensor mounted on footwear, a mounting-direction-determination unit that determines a mounting direction of the sensor using the acquired sensor data, a coordinate conversion unit that converts a coordinate system of the sensor data in accordance with the determined mounting direction of the sensor, a detection unit that detects a walking event from time-series data of the sensor data in which the coordinate system has been converted, a calculation unit that calculates a gait parameter in accordance with the detected walking event, and a transmission unit that transmits the calculated gait parameter.

In a gait measurement method of one aspect of the present disclosure, sensor data measured by a sensor mounted on footwear is acquired, a mounting direction of the sensor is determined using the acquired sensor data, a coordinate system of the sensor data is converted in accordance with the determined mounting direction of the sensor, a walking event is detected from time-series data of the sensor data in which the coordinate system has been converted, a gait parameter is calculated in accordance with the detected walking event, and the calculated gait parameter is transmitted.

A program of one aspect of the present disclosure allows a computer to execute processing of acquiring sensor data measured by a sensor mounted on footwear, processing of determining a mounting direction of the sensor using the acquired sensor data, processing of converting a coordinate system of the sensor data in accordance with the determined mounting direction of the sensor, processing of detecting a walking event from time-series data of the sensor data in which the coordinate system has been converted, processing of calculating a gait parameter in accordance with the detected walking event, and processing of transmitting the calculated gait parameter.

### Advantageous Effects of Invention

According to the present disclosure, it is possible to provide the gait measurement device and the like capable of measuring the sensor data relevant to the motion of the foot regardless of the mounting direction of the sensor.

### BRIEF DESCRIPTION OF DRAWINGS

FIG. 1 is a block diagram illustrating an example of a configuration of a measurement device according to a first example embodiment.

FIG. 2 is a conceptual diagram illustrating a mounting example of the measurement device according to the first example embodiment.

FIG. 3 is a conceptual diagram for illustrating a coordinate system relevant to the measurement device according to the first example embodiment.

FIG. 4 is a conceptual diagram for illustrating a human body surface to be a reference of sensor data measured by the measurement device according to the first example embodiment.

FIG. 5 is a conceptual diagram for illustrating a walking event detected by the measurement device according to the first example embodiment.

FIG. 6 is a conceptual diagram for illustrating an example of a change in a local coordinate system by rotating the measurement device according to the first example embodiment around an axis in a vertical direction.

FIG. 7 is a conceptual diagram for illustrating a conversion matrix for rotating the measurement device according to the first example embodiment around the axis in the vertical direction.

FIG. 8 is a conceptual diagram for illustrating another example in which the local coordinate system of the measurement device according to the first example embodiment is rotated around the axis in the vertical direction.

FIG. 9 is a conceptual diagram for illustrating a conversion matrix for rotating the measurement device according to the first example embodiment around an axis in a right-left direction and a front-back direction.

FIG. 10 is a table for showing a conversion table used for conversion of the local coordinate system of the measurement device according to the first example embodiment.

FIG. 11 is a conceptual diagram for illustrating an example of a conversion formula for converting the local coordinate system of the measurement device according to the first example embodiment.

FIG. 12 is a flowchart illustrating an example of an operation of the measurement device according to the first example embodiment.

FIG. 13 is a flowchart for illustrating an example of measurement preparation processing included in the operation of the measurement device according to the first example embodiment.

FIG. 14 is a flowchart for illustrating an example of gait parameter calculation processing included in the operation of the measurement device according to the first example embodiment.

FIG. 15 is a block diagram illustrating an example of a configuration of a measurement device according to a second example embodiment.

FIG. 16 is a conceptual diagram illustrating an example of displaying information output from a gait measurement system according to the second example embodiment on a screen of a mobile terminal.

FIG. 17 is a block diagram illustrating an example of a configuration of a gait measurement device according to a third example embodiment.

FIG. 18 is a block diagram illustrating an example of a hardware configuration that executes control and processing according to each of the example embodiments.

### EXAMPLE EMBODIMENT

Hereinafter, example embodiments of the present invention will be described with reference to the drawings. However, the example embodiments described below have technically preferable limitations for carrying out the present invention, but the scope of the invention is not limited to the following. In all the drawings used for the following description of the example embodiments, the same reference numerals will be applied to the same parts unless there is a particular reason. In the following example embodiments, the repeated description of similar configurations and operations may be omitted.

### First Example Embodiment

First, a measurement device according to a first example embodiment will be described with reference to the drawings. The measurement device of the present example embodiment measures a feature (also referred to as a gait) included in a walking pattern of a user by using sensor data measured by a sensor mounted on the footwear of the user. The measurement device of the present example embodiment determines the mounting direction of the sensor using the sensor data. The measurement device according to the present example embodiment converts the local coordinate system of the sensor in accordance with the determined mounting direction of the sensor. Hereinafter, an example will be described in which the sensor is incorporated in the measurement device. The sensor may be configured as hardware different from the measurement device.

### (Configuration)

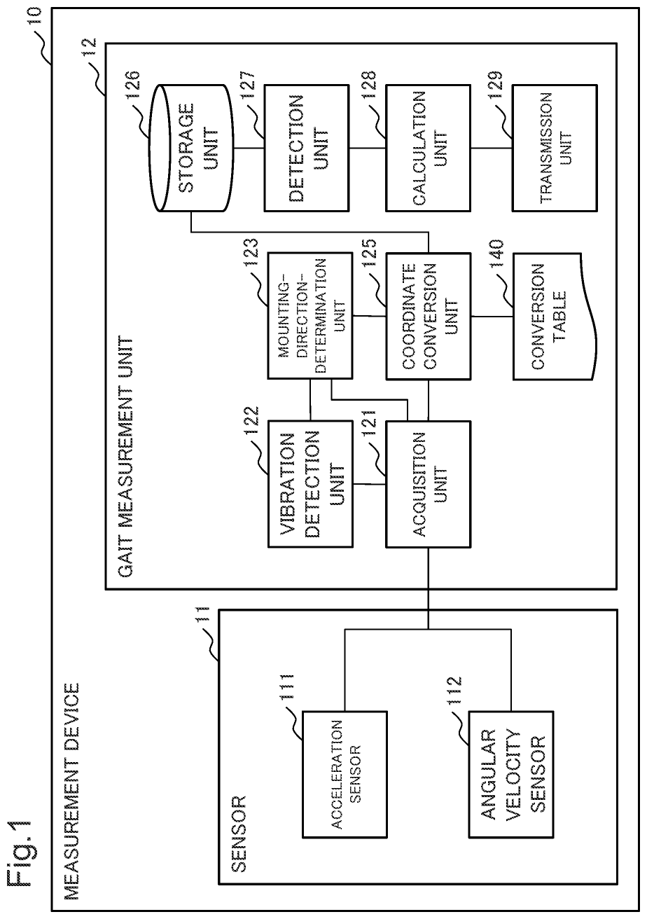

FIG. 1 is a block diagram illustrating an example of the configuration of a measurement device 10 according to the present example embodiment. The measurement device 10 includes a sensor 11 and a gait measurement unit 12. In the present example embodiment, the sensor 11 and the gait measurement unit 12 are configured as a single package. For example, the sensor 11 and the gait measurement unit 12 may be configured as an individual package. For example, the measurement device 10 may be configured only by the gait measurement unit 12 by excluding the sensor 11 from the configuration of the measurement device 10. The measurement device 10 is installed on the foot portion. For example, the measurement device 10 is installed on footwear such as shoes. In the present example embodiment, an example will be described in which the measurement device 10 is mounted at a position on the back side of the arch of the foot. Hereinafter, the configuration of the sensor 11 and the gait measurement unit 12 will be individually described.

### [Sensor]

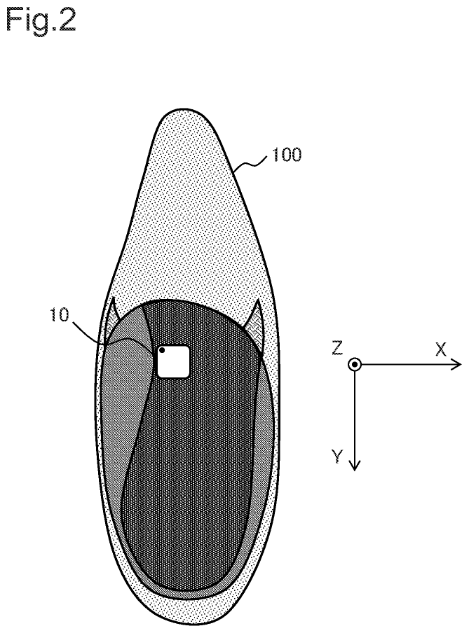

The sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. The sensor other than the acceleration sensor 111 and the angular velocity sensor 112 that can be included in the sensor 11 will not be described.

The acceleration sensor 111 is a sensor that measures an acceleration (also referred to as a spatial acceleration) in three axial directions. The acceleration sensor 111 measures the acceleration (also referred to as the spatial acceleration) as a physical amount relevant to the motion of the foot. The acceleration sensor 111 outputs the measured acceleration to the gait measurement unit 12. For example, a sensor of a piezoelectric type, a piezoresistive type, a capacitance type, or the like can be used as the acceleration sensor 111. The sensor used as the acceleration sensor 111 is not limited to the measurement method as long as the sensor is capable of measuring the acceleration.

The angular velocity sensor 112 is a sensor that measures an angular velocity (also referred to as a spatial angular velocity) around three axes. The angular velocity sensor 112 measures the angular velocity (also referred to as the spatial angular velocity) as the physical amount relevant to the motion of the foot. The angular velocity sensor 112 outputs the measured angular velocity to the gait measurement unit 12. For example, a sensor of a vibration type, a capacitance type, or the like can be used as the angular velocity sensor 112. The sensor used as the angular velocity sensor 112 is not limited to the measurement method as long as the sensor is capable of measuring the angular velocity.

The sensor 11 is enabled by, for example, an inertial measurement device that measures the acceleration and the angular velocity. An example of the inertial measurement device is an inertial measurement unit (IMU). The IMU includes an acceleration sensor that measures an acceleration in three axial directions and an angular velocity sensor that measures an angular velocity around three axes. The sensor 11 may be enabled by an inertial measurement device such as vertical gyro (VG) or an attitude heading (AHRS). The sensor 11 may be enabled by global positioning system/inertial navigation system (GPS/INS). The sensor 11 may be enabled by a device other than the inertial measurement device as long as the device is capable of measuring the physical amount relevant to the motion of the foot.

FIG. 2 is a conceptual diagram illustrating an example in which the measurement device 10 is mounted in a shoe 100. In the example of FIG. 2, the measurement device 10 is installed at a position on the back side of the arch of foot. For example, the measurement device 10 is mounted on an insole inserted into the shoe 100. For example, the measurement device 10 may be mounted on the bottom surface of the shoe 100. For example, the sensor 11 may be embedded in the main body of the shoe 100. The measurement device 10 may be detachable from the shoe 100 or may not be detachable from the shoe 100. The measurement device 10 may be installed at a position other than the back side of the arch of the foot as long as sensor data relevant to the motion of the foot can be acquired. The measurement device 10 may be installed on a sock worn by the user or a decorative article such as an anklet worn by the user. The measurement device 10 may be directly attached to the foot or embedded in the foot.

FIG. 2 illustrates an example in which the measurement device 10 is installed in the shoe 100 on the right foot side. The measurement device 10 may be installed in the shoe 100 on the left foot side. The measurement device 10 may be installed in the shoes 100 of both feet. In a case where the measurement device 10 is installed on the shoes 100 of both feet, the gait of the user can be measured based on the motion of both feet. In the present example embodiment, a system will be described in which the right foot is a reference foot and the left foot is an opposite foot. The method of the present example embodiment can also be applied to a system in which the left foot is a reference foot and the right foot is an opposite foot.

FIG. 2 illustrates an example in which the mounting direction of the measurement device 10 (sensor 11) is normal. The normal mounting direction is also referred to as a first mounting direction. The local coordinate system in the normal mounting direction is also referred to as a local coordinate system in the first mounting direction. In FIG. 2, as a mark of the mounting direction, a dot is formed on the upper left of a first surface of the measurement device 10. When the mounting direction is normal, the measurement device 10 is mounted such that the first surface is directed upward (in a +Z direction). That is, when the mounting direction is normal, the measurement device 10 is mounted such that a second surface facing the first surface is directed downward (in a −Z direction). The measurement device 10 may take four mounting directions of 0 degrees, 90 degrees, 180 degrees, and 270 degrees clockwise around the z axis with a case where the mounting direction is normal as a reference (0 degrees). The measurement device 10 may take two mounting directions of a case where the first surface is mounted upward and a case where the first surface is mounted downward. That is, in the present example embodiment, there are 8 mounting directions of the sensor 11. The details of the mounting direction of the sensor 11 will be described later. In the present example embodiment, it is assumed that the mounting directions of the measurement device 10 and the sensor 11 coincide with each other. When the mounting directions of the measurement device 10 and the sensor 11 do not coincide with each other, the mounting direction of the measurement device 10 is determined in accordance with the mounting direction of the sensor 11.

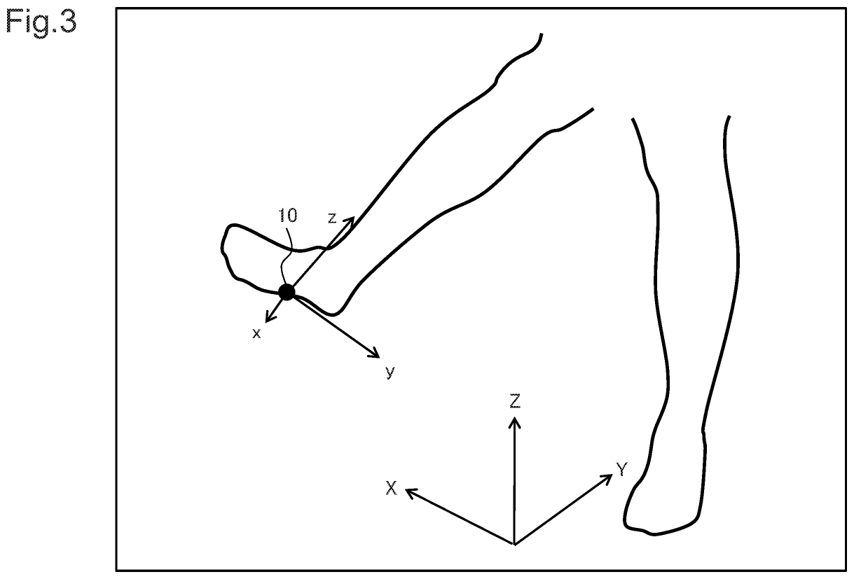

FIG. 3 is a conceptual diagram for illustrating a local coordinate system (x axis, y axis, z axis) set in the measurement device 10 and a world coordinate system (X axis, Y axis, Z axis) set with respect to the ground in a case where the measurement device 10 is installed on the back side of the arch of foot. FIG. 3 illustrates an example in which the mounting direction of the sensor 11 is normal. In the world coordinate system, the horizontal direction of the user is set to an X-axial direction (a rightward direction is positive) while the user is standing upright. In the world coordinate system, the traveling direction of the user is set to a Y-axial direction (a backward direction is positive). In the world coordinate system, the vertical direction is set to a Z-axial direction (a vertically upward direction is positive). In the present example embodiment, a local coordinate system including an x direction, a y direction, and a z direction based on the measurement device 10 is set. The local coordinate system set in the measurement device 10 is not limited to the example of FIG. 3.

In the present example embodiment, the direction of the local coordinate system (x axis, y axis, z axis) set in the measurement device 10 is changed in accordance with the mounting direction of the sensor 11 in the shoe 100. Therefore, in any mounting direction, the x axis is referred to as a first axis (a front-back axis), the y axis is referred to as a second axis (a right-left axis), and the z axis is referred to as a third axis (a vertical axis) so that the direction of the local coordinate system can be distinguished. The first axis is an axis along a right-left axial direction (the x direction). The second axis is an axis along a front-back axial direction (the y direction). The third axis is an axis along a vertical axial direction (the z direction).

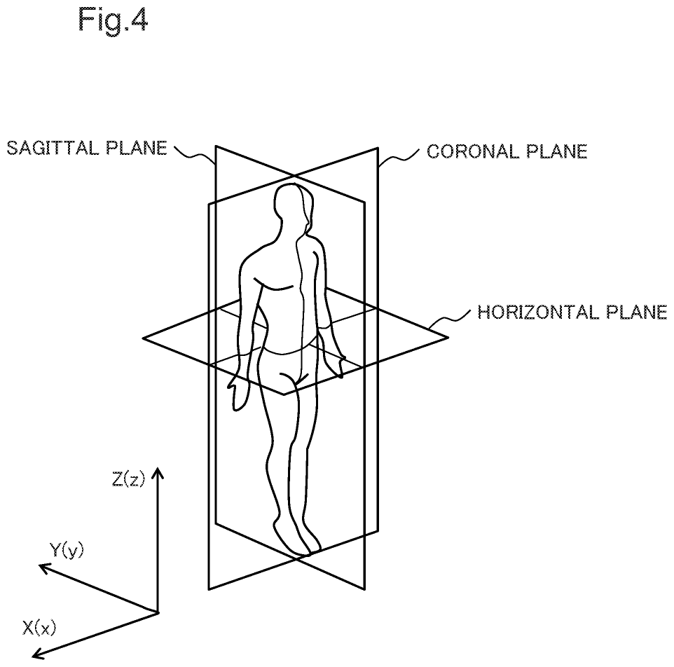

FIG. 4 is a conceptual diagram for illustrating a surface set for the human body (also referred to as a human body surface). In the present example embodiment, a sagittal plane dividing the body into right and left, a coronal plane dividing the body into front and back, and a horizontal plane dividing the body horizontally are defined. In the example of FIG. 4, it is assumed that the world coordinate system and the local coordinate system coincide with each other in an upright state. In the present example embodiment, a rotation in the sagittal plane with the x axis as a rotation axis is defined as roll, a rotation in the coronal plane with the y axis as a rotation axis is defined as pitch, and a rotation in the horizontal plane with the z axis as a rotation axis is defined as yaw. A rotation angle in a sagittal plane with the x axis as a rotation axis is defined as a roll angle, a rotation angle in the coronal plane with the y axis as a rotation axis is defined as a pitch angle, and a rotation angle in the horizontal plane with the z axis as a rotation axis is defined as a yaw angle.

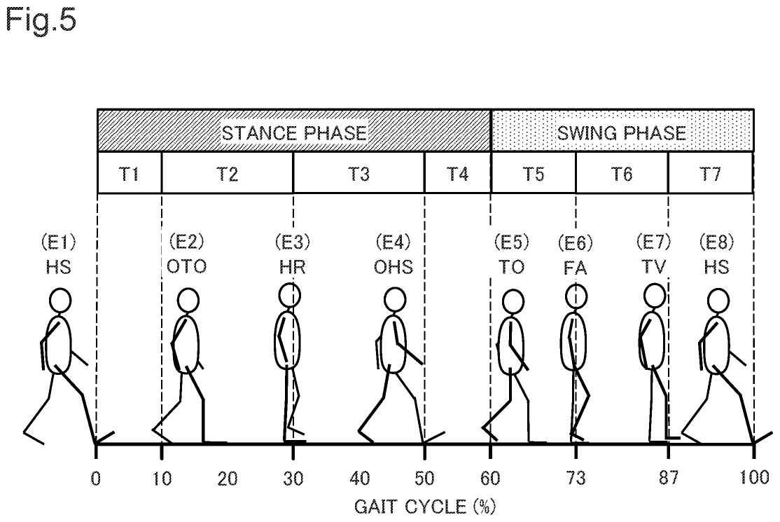

FIG. 5 is a conceptual diagram for illustrating a walking event detected in one gait cycle with the right foot as a reference. The horizontal axis in FIG. 5 is a gait cycle normalized with one gait cycle of the right foot as 100 percent (%) in which a time point when the heel of the right foot lands on the ground is set as a starting point and a time point when the heel of the right foot next lands on the ground is set as an ending point. The one gait cycle of one foot is roughly divided into a stance phase in which at least a part of the back side of the foot is in contact with the ground and a swing phase in which the back side of the foot is separated from the ground. In the example of FIG. 5, normalization is performed such that the stance phase occupies 60% and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. A walking waveform for one gait cycle may not start from the time point when the heel lands on the ground. For example, the start point of the walking waveform for one gait cycle may be set at the central time point of the stance phase.

In FIG. 5, a walking event E1 represents an event (heel strike: HS) in which the heel of the right foot touches the ground. A walking event E2 represents an event (opposite toe off: OTO) in which the toe of the left foot is separated from the ground while the ground contact surface of the sole of the right foot is in contact with the ground. A walking event E3 represents an event (heel rise: HR) in which the heel of the right foot rises while the ground contact surface of the sole of the right foot is in contact with the ground. A walking event E4 is an event (opposite heel strike: OHS) in which the heel of the left foot is in contact with the ground. A walking event E5 represents an event (toe off: TO) in which the toe of the right foot is separated from the ground while the ground contact surface of the sole of the left foot is in contact with the ground. A walking event E6 represents an event (foot adjacent: FA) in which the left foot and the right foot intersect with each other while the ground contact surface of the sole of the left foot is in contact with the ground. A walking event E7 represents an event (tibia vertical: TV) in which the tibia of the right foot is approximately perpendicular to the ground while the sole of the left foot is in contact with the ground. A walking event E8 represents an event (heel strike: HS) in which the heel of the right foot is in contact with the ground. The walking event E8 is associated with the end point of the gait cycle starting from the walking event E1 and associated with the start point of the next gait cycle.

### [Gait Measurement Unit]

As illustrated in FIG. 1, the gait measurement unit 12 (also referred to as a gait measurement device) includes an acquisition unit 121, a vibration detection unit 122, a mounting-direction-determination unit 123, a coordinate conversion unit 125, a storage unit 126, a detection unit 127, a calculation unit 128, and a transmission unit 129. The gait measurement unit 12 includes a conversion table 140 for converting the local coordinate system of the sensor data measured by the sensor 11 in accordance with the mounting direction of the sensor 11. The gait measurement unit 12 is operated in three modes of a vibration detection mode, a stable walking determination mode, and a measurement mode.

The gait measurement unit 12 is enabled by a microcomputer or a microcontroller. For example, the gait measurement unit 12 includes a control circuit and a storage circuit. For example, the control circuit is enabled by a central processing unit (CPU). For example, the storage circuit is enabled by a volatile memory such as a random access memory (RAM). For example, the storage circuit is enabled by a non-volatile memory such as a read only memory (ROM) or an electrically erasable and programmable read only memory (EEPROM).

The acquisition unit 121 acquires sensor data measured in accordance with the walking of the user from the sensor 11. For example, the acquisition unit 121 performs analog-to-digital conversion (AD conversion) on the acquired physical amount (analog data) such as the angular velocity and the acceleration. The physical amount (the analog data) measured by the acceleration sensor 111 and the angular velocity sensor 112 may be converted into digital data in each of the acceleration sensor 111 and the angular velocity sensor 112.

In the vibration detection mode, the acquisition unit 121 acquires a vertical-direction acceleration (a z-direction acceleration) from the sensor 11. The vibration detection mode is a low-power mode for measuring only the vertical-direction acceleration (the z-direction acceleration). The acquisition unit 121 outputs the acquired sensor data (the vertical-direction acceleration) to the vibration detection unit 122.

The vibration detection unit 122 acquires the sensor data (the vertical-direction acceleration) from the acquisition unit 121 in the vibration detection mode. The vibration detection unit 122 detects a vibration in accordance with the value of the vertical-direction acceleration (the z-direction acceleration). In a case where the value of the vertical-direction acceleration (the z-direction acceleration) exceeds a first threshold value (a), the vibration detection unit 122 determines that walking has started. In a case where it is determined that walking has started, the vibration detection unit 122 outputs a mounting-direction-determination instruction to the mounting-direction-determination unit 123. The acquisition unit 121 outputs the sensor data (the vertical-direction acceleration) to the mounting-direction-determination unit 123. When the operation mode of the measurement device 10 does not include the vibration detection mode, the vibration detection unit 122 may be omitted.

The mounting-direction-determination unit 123 acquires the mounting-direction-determination instruction from vibration detection unit 122. The mounting-direction-determination unit 123 acquires the sensor data (the vertical-direction acceleration) from the vibration detection unit 122. The mounting-direction-determination unit 123 may acquire the sensor data (the vertical-direction acceleration) from the acquisition unit 121 in accordance with the acquisition of the mounting-direction-determination instruction. In accordance with the acquisition of the mounting-direction-determination instruction, the mounting-direction-determination unit 123 determines the mounting direction of the sensor 11 using the sensor data (the vertical-direction acceleration). The mounting-direction-determination unit 123 determines the mounting direction (the both surfaces) of the sensor 11, and then determines the mounting direction (the rotation) of the sensor 11 around the third axis (the z axis).

First, the mounting-direction-determination unit 123 determines the mounting direction (the both surfaces) of the sensor 11. When the mounting direction is normal, the measurement device 10 is mounted such that the first surface is directed upward (the +Z direction). On the other hand, the measurement device 10 may be mounted in a reversed manner such that the first surface is directed downward (the −Z direction). Therefore, the mounting-direction-determination unit 123 performs threshold value determination on the first threshold value set to the vertical-direction acceleration (the Z-direction acceleration) based on two criteria. First, when the value of the vertical-direction acceleration (the Z-direction acceleration) exceeds a value (1G+α) obtained by adding the first threshold value (a) to the gravitational acceleration 1G, the mounting-direction-determination unit 123 determines that the first surface of the measurement device 10 is mounted upward (+Z). Second, when the value of the vertical-direction acceleration (the Z-direction acceleration) falls below a value (−1G−α) obtained by multiplying the value obtained by adding the first threshold value (α) to the gravitational acceleration 1G by −1, the mounting-direction-determination unit 123 determines that the first surface of the measurement device 10 is mounted downward (−Z). The value (−1G−α) obtained by multiplying the value obtained by adding the first threshold value (α) to the gravitational acceleration 1G by −1 is also referred to as a negative value. In this manner, the mounting-direction-determination unit 123 determines the mounting direction (the both surfaces) of the sensor 11 by determining the threshold values of two systems with respect to the vertical-direction acceleration (the Z-direction acceleration).

When determining the mounting direction (the both surfaces) of the sensor 11, the gait measurement unit 12 shifts to the stable walking determination mode. The stable walking determination mode is a normal-power mode in which all the spatial accelerations/spatial angular velocities are continuously measured. At the timing of shifting to the stable walking determination mode, the gait measurement unit 12 activates a CPU (not illustrated) that controls the sensor 11. Upon activation, the CPU controls the sensor 11 to start the continuous measurement of all spatial accelerations/spatial angular velocities.

In accordance with the shift to the stable walking determination mode, the acquisition unit 121 acquires the acceleration in the three axial directions and the angular velocity around the three axes measured by the acceleration sensor 111 and the angular velocity sensor 112 included in the sensor 11. The acquisition unit 121 outputs the acquired acceleration in the three axial directions and the acquired angular velocity around the three axes to the mounting-direction-determination unit 123 and the measurement unit 124. The acquisition unit 121 may be configured to output only a first-axial-direction acceleration (an x-direction acceleration) and a second-axial-direction acceleration (a y-direction acceleration) to the mounting-direction-determination unit 123.

In accordance with the shift to the stable walking determination mode, the mounting-direction-determination unit 123 acquires the acceleration in the three axial directions and the angular velocity around the three axes from the acquisition unit 121. The mounting-direction-determination unit 123 may be configured to acquire only the first-axial-direction acceleration (the x-direction acceleration) and the second-axial-direction acceleration (the y-direction acceleration) from acquisition unit 121. The mounting-direction-determination unit 123 determines the mounting direction (the rotation) of the sensor 11 around the third axis (the z axis) using the first-axial-direction acceleration (the x-direction acceleration) and the second-axial-direction acceleration (the y-direction acceleration).

When the mounting direction of the sensor 11 is normal (+z is the upward direction, ±y is the backward direction), the traveling direction (the Y direction) coincides with the second axis (the y direction). In this case, the mounting-direction-determination unit 123 may determine that the stable walking has started when the second-axial-direction acceleration (the y-direction acceleration) exceeds a second threshold value (B). When the upper and lower mounting directions of the measurement device 10 are normal and the front and back mounting directions are opposite to each other (+z is the upward direction, ±y is the forward direction), the traveling direction (the Y direction) coincides with the second axis (the y direction), but the second axis (the y axis) is opposite in positive and negative. In general, the acceleration in the traveling direction (the Y direction) is larger in a forward acceleration (a −Y direction) than in a backward acceleration (+Y). Therefore, the mounting-direction-determination unit 123 determines the axial direction with the maximum acceleration as the −y direction. Since a positional relationship between the first axis (the x axis) and the second axis (the y axis) is determined, in a case where the −y direction is determined, the mounting direction of the sensor 11 can be determined.

When the mounting direction of the sensor 11 is unknown, the mounting-direction-determination unit 123 is not capable of determining the mounting direction of the sensor 11 only by the second-axial-direction acceleration (the y-direction acceleration). Therefore, the mounting-direction-determination unit 123 determines the mounting direction using the absolute value of the first-axial-direction acceleration (the x-direction acceleration) and the absolute value of the second-axial-direction acceleration (the y-direction acceleration). In general, the maximum value of the absolute value of a traveling-direction acceleration (a Y-direction acceleration) is a value close to three times the maximum value of the absolute value of a right-left direction acceleration (an X-direction acceleration). For example, when a ratio of the larger value to the smaller value among the maximum values of the absolute values in the first axial direction (the x direction) and the second axial direction (the y direction) exceeds a third threshold value (γ), the mounting-direction-determination unit 123 determines that the axial direction indicating the larger value is along the traveling direction (the Y axis). For example, the third threshold value (γ) is set to 3. With regard to the axial direction determined to be along the traveling direction (the Y axis), the mounting-direction-determination unit 123 determines the axial direction with a larger maximum value of the acceleration as the −y direction. A direction in which the absolute values of the first axial direction (the x direction) and the second axial direction (the y direction) are maximum is associated with the forward direction (the −y direction). Therefore, the mounting-direction-determination unit 123 may determine that the direction in which the absolute values of the first axial direction (the x direction) and the second axial direction (the y direction) are maximum is the forward direction (the −y direction). As described above, since the positional relationship between the first axis (the x axis) and the second axis (the y axis) is determined, in a case where the −y direction is determined, the mounting direction of the sensor 11 can be determined.

The mounting-direction-determination unit 123 outputs the mounting direction of the sensor 11 to the coordinate conversion unit 125. The coordinate conversion unit 125 converts the local coordinate system of the sensor data measured by the sensor 11 into the local coordinate system in the normal mounting direction (the first mounting direction) in accordance with the mounting direction of the sensor 11 determined by the mounting-direction-determination unit 123. Such conversion is equivalent to converting the coordinates of the sensor data measured by the sensor 11 into the local coordinate system in the normal mounting direction.

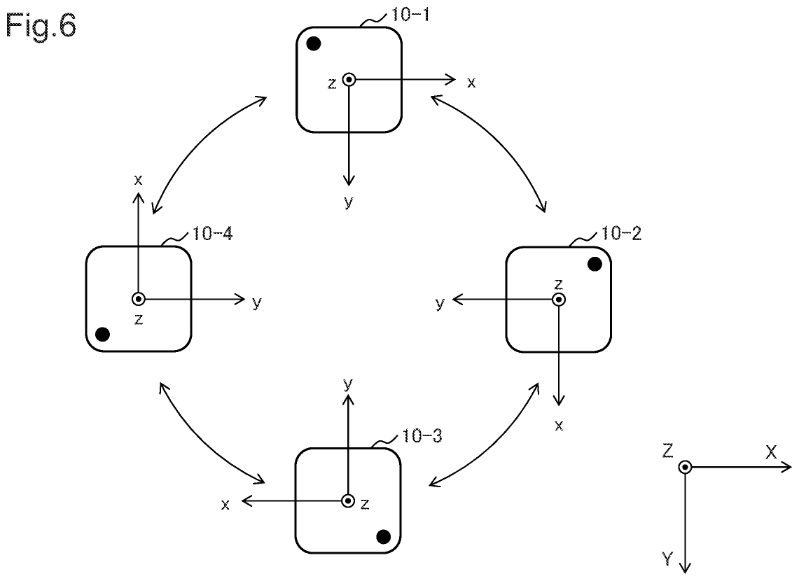

FIG. 6 is a conceptual diagram for illustrating the mounting direction (the rotation) of the sensor 11 around the third axis (the z axis) when the both surfaces of the measurement device 10 are normally mounted. FIG. 6 illustrates an example in which the both surfaces of the measurement device 10 are normally mounted. The lower right side in FIG. 6 illustrates the world coordinate system (X, Y, Z). When the both surfaces of the measurement device 10 are normally mounted, there are four mounting directions (rotations) of the sensor 11. In FIG. 6, measurement devices 10-1 to 4 are used to distinguish the mounting direction (the rotation) of the sensor 11.

The measurement device 10-1 (the upper side in FIG. 6) is in a normal mounting direction (rotation). The local coordinate system of the measurement device 10-1 (the upper side in FIG. 6) coincides with the world coordinate system.

The measurement device 10-2 (the right side in FIG. 6) is in a state of being rotated clockwise by 90 degrees around the third axis (the z axis) from the normal mounting direction (rotation). The local coordinate system of the measurement device 10-2 (the right side in FIG. 6) coincides with the world coordinate system when rotated counterclockwise by 90 degrees around the third axis (the z axis).

The measurement device 10-3 (the lower side in FIG. 6) is rotated by 180 degrees around the third axis (the z axis) from the normal mounting direction (rotation). The local coordinate system of the measurement device 10-3 (the lower side in FIG. 6) coincides with the world coordinate system when rotated by 180 degrees around the third axis (the z axis).

The measurement device 10-4 (the left side in FIG. 6) is in a state of being rotated counterclockwise by 90 degrees around the third axis (the z axis) from the normal mounting direction (rotation). The local coordinate system of the measurement device 10-4 (the left side in FIG. 6) coincides with the world coordinate system when rotated clockwise by 90 degrees around the third axis (the z axis).

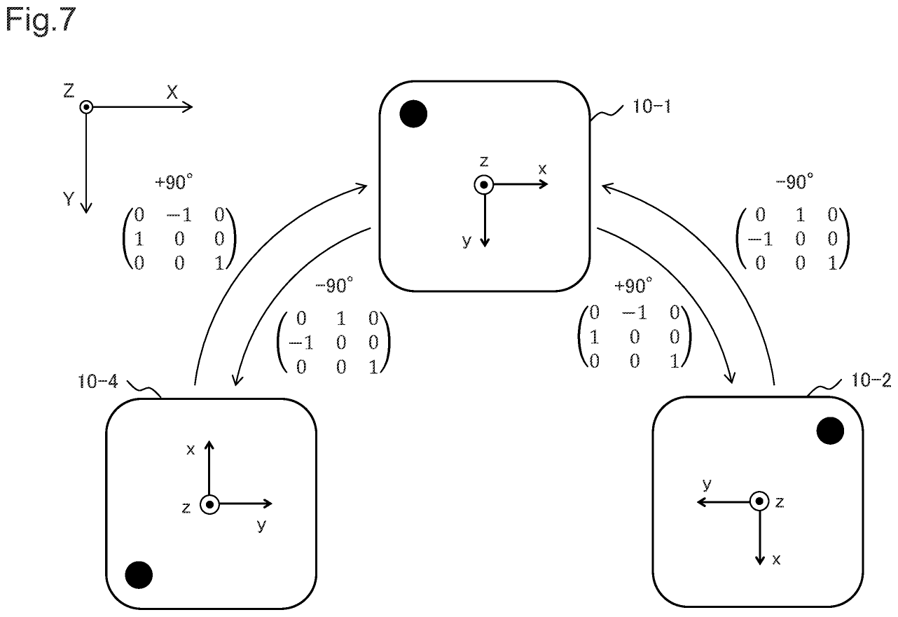

FIG. 7 is a conceptual diagram for illustrating the rotation of the measurement device 10 around the third axis (the z axis). In FIG. 7, a clockwise rotation is positive. FIG. 7 illustrates a rotation matrix for rotating the measurement device by +90 degrees (also referred to as a first rotation matrix R₁) and a rotation matrix for rotating the measurement device by −90 degrees (also referred to as a second rotation matrix R₂). The first rotation matrix R₁ and the second rotation matrix R₂ are as follows.

$$\begin{matrix}[ Formula 1 ] & \\ {R}_{1} = ( \begin{matrix}0 & - 1 & 0 \\ 1 & 0 & 0 \\ 0 & 0 & 1\end{matrix} ) & ( 1 )\end{matrix}$$

$$\begin{matrix}[ Formula 2 ] & \\ {R}_{2} = ( \begin{matrix}0 & 1 & 0 \\ - 1 & 0 & 0 \\ 0 & 0 & 1\end{matrix} ) & ( 2 )\end{matrix}$$

The above Formulae 1 and 2 may be used in accordance with the positive and negative definitions of the rotation direction.

When the coordinates of the measurement device 10-1 (the upper side in FIG. 7) are multiplied by the first rotation matrix R₁, the coordinates are converted into the local coordinate system of the measurement device 10-2 (the right side in FIG. 7). On the other hand, when the coordinates of the measurement device 10-2 (the right side in FIG. 7) are multiplied by the second rotation matrix R₂, the coordinates are converted into the local coordinate system of the measurement device 10-1 (the upper side in FIG. 7). That is, in order to convert the coordinates of the measurement device 10-2 (the right side in FIG. 7) into the local coordinate system of the measurement device 10-1 (the upper side in FIG. 7), the coordinates of the measurement device 10-2 (the right side in FIG. 7) may be multiplied by the second rotation matrix R₂.

When the coordinates of the measurement device 10-1 (the upper side in FIG. 7) are multiplied by the second rotation matrix R₂, the coordinates are converted into the local coordinate system of the measurement device 10-4 (the left side in FIG. 7). On the other hand, when the coordinates of the measurement device 10-4 (the left side in FIG. 7) are multiplied by the first rotation matrix R₁, the coordinates are converted into the local coordinate system of the measurement device 10-1 (the upper side in FIG. 7). That is, in order to convert the coordinates of the measurement device 10-4 (the left side in FIG. 7) into the local coordinate system of the measurement device 10-1 (the upper side in FIG. 7), the coordinates of the measurement device 10-4 (the left side in FIG. 7) may be multiplied by the first rotation matrix R₁.

In FIG. 6, in order to convert the coordinates of the measurement device 10-3 (the lower side in FIG. 6) into the local coordinate system of the measurement device 10-1 (the upper side in FIG. 6), the coordinates of the measurement device 10-3 (the lower side in FIG. 6) may be multiplied twice by the first rotation matrix R₁. In order to convert the coordinates of the measurement device 10-3 (the lower side in FIG. 6) into the local coordinate system of the measurement device 10-1 (the upper side in FIG. 6), the second rotation matrix R₂ may be multiplied twice by the local coordinate system of the measurement device 10-3 (the lower side). Even in a case where the first rotation matrix R₁ is multiplied twice or the second rotation matrix R₂ is multiplied twice, a rotation matrix of Formula 3 described below is obtained.

$$\begin{matrix}[ Formula 3 ] & \\ {R}_{3} = ( \begin{matrix}- 1 & 0 & 0 \\ 0 & - 1 & 0 \\ 0 & 0 & 1\end{matrix} ) & ( 3 )\end{matrix}$$

The above formula can be used regardless of whether the rotation direction is positive or negative.

The rotation matrix of Formula 3 described above is a rotation matrix (also referred to as a third rotation matrix R₃) for rotating the measurement device by +180 degrees (−180 degrees). That is, when the coordinates of the measurement device 10-3 (the lower side in FIG. 6) are multiplied by the third rotation matrix, the coordinates are converted into the local coordinate system of the measurement device 10-1 (the upper side in FIG. 6). In FIG. 7, the third rotation matrix R₃ is omitted.

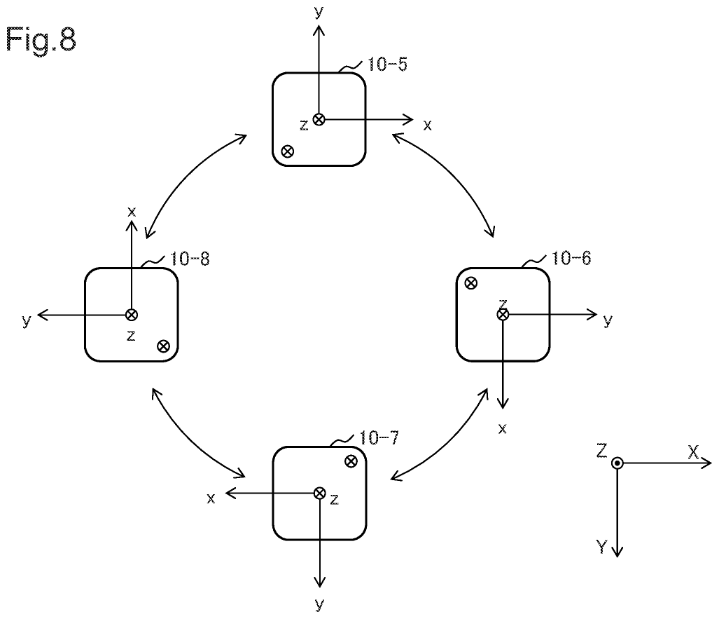

FIG. 8 is a conceptual diagram for illustrating the mounting direction (the rotation) of the sensor 11 around the third axis (the z axis) in a case where the both surfaces of the measurement device 10 are mounted opposite to each other. FIG. 8 illustrates an example in which the both surfaces of the measurement device 10 are mounted opposite to each other. The lower right side in FIG. 8 illustrates the world coordinate system (X, Y, Z). When the both surfaces of the measurement device 10 are mounted opposite to each other, there are four mounting directions (rotations) of the sensor 11. In FIG. 8, measurement devices 10-5 to 8 are used to distinguish the mounting direction (the rotation) of the sensor 11.

The measurement device 10-5 (the upper side in FIG. 8) is in a state of being rotated by 180 degrees around the first axis (the x axis) from the normal mounting direction (the rotation). The local coordinate system of the measurement device 10-5 (the upper side in FIG. 8) coincides with the world coordinate system when rotated by 180 degrees around the first axis (the x axis).

The measurement device 10-6 (the right side in FIG. 8) is in a state of being rotated clockwise by 90 degrees around the third axis (the z axis) from the mounting direction (the rotation) of the measurement device 10-5 (the upper side in FIG. 8). The local coordinate system of the measurement device 10-5 (the right side in FIG. 8) coincides with the world coordinate system when rotated counterclockwise by 90 degrees around the third axis (the z axis) and rotated by 180 degrees around the first axis (the x axis).

The measurement device 10-7 (the lower side in FIG. 8) is in a state of being rotated by 180 degrees around the third axis (the z axis) from the mounting direction (the rotation) of the measurement device 10-5 (the upper side in FIG. 8). The local coordinate system of the measurement device 10-7 (the lower side in FIG. 8) coincides with the world coordinate system when rotated by 180 degrees around the third axis (the z axis) and rotated by 180 degrees around the first axis (the x axis).

The measurement device 10-8 (the left side in FIG. 8) is in a state of being rotated counterclockwise by 90 degrees around the third axis (the z axis) from the mounting direction (the rotation) of the measurement device 10-5 (the upper side in FIG. 8). The local coordinate system of the measurement device 10-8 (the left side) coincides with the world coordinate system when rotated clockwise by 90 degrees around the third axis (the z axis) and rotated by 180 degrees around the first axis (the x axis).

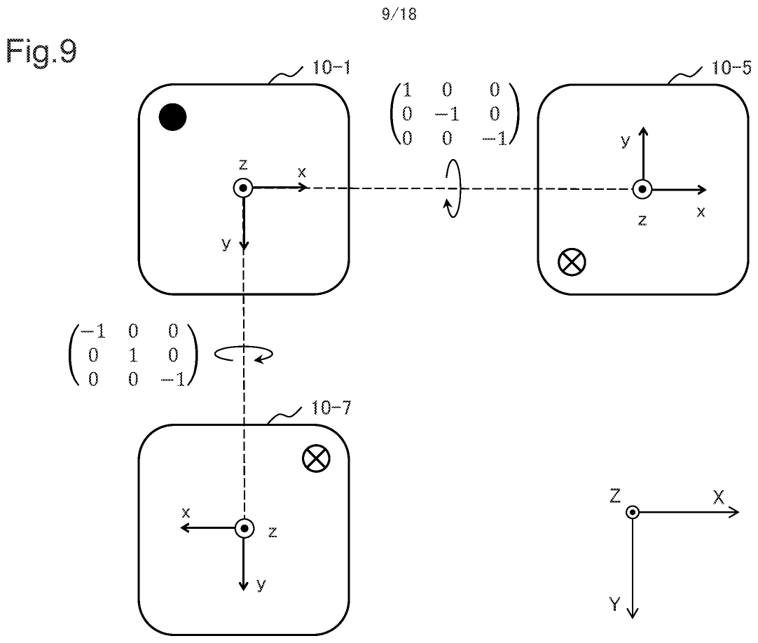

FIG. 9 is a conceptual diagram for illustrating the rotation of the measurement device 10 around the first axis (the x axis) and the second axis (the y axis). FIG. 9 illustrates a rotation matrix (also referred to as a fourth rotation matrix R₄) rotated around the first axis (the x axis) by 180 degrees and a rotation matrix (also referred to as a fifth rotation matrix R₅) rotated around the second axis (the y axis) by 180 degrees. The fourth rotation matrix R₄ and the fifth rotation matrix R₅ are as follows.

$$\begin{matrix}[ Formula 4 ] & \\ {R}_{4} = ( \begin{matrix}1 & 0 & 0 \\ 0 & - 1 & 0 \\ 0 & 0 & - 1\end{matrix} ) & ( 4 )\end{matrix}$$

$$\begin{matrix}[ Formula 5 ] & \\ {R}_{4} = ( \begin{matrix}- 1 & 0 & 0 \\ 0 & 1 & 0 \\ 0 & 0 & - 1\end{matrix} ) & ( 5 )\end{matrix}$$

Formulae 4 and 5 described above may be used in accordance with the positive and negative definitions of the rotation direction.

When the coordinates of the measurement device 10-1 (the upper left side in FIG. 9) are multiplied by the fourth rotation matrix R₄, the coordinates are converted into the local coordinate system of the measurement device 10-5 (the upper right side in FIG. 9). When the coordinates of the measurement device 10-5 (the upper right side in FIG. 9) are multiplied by the fourth rotation matrix R₄, the coordinates are converted into the local coordinate system of the measurement device 10-1 (the upper left side in FIG. 9). That is, in order to convert the coordinates of the measurement device 10-5 (the upper right side in FIG. 9) into the local coordinate system of the measurement device 10-1 (the upper left side in FIG. 9), the coordinates of the measurement device 10-5 (the upper right side in FIG. 9) may be multiplied by the fourth rotation matrix R₄.

By multiplying the coordinates of the measurement device 10-1 (the upper left side in FIG. 9) by the fifth rotation matrix R₅, the coordinates are converted into the local coordinate system of the measurement device 10-7 (the lower left side in FIG. 9). When the coordinates of the measurement device 10-7 (the lower left side in FIG. 9) are multiplied by the fifth rotation matrix R₅, the coordinate is converted into the local coordinate system of the measurement device 10-1 (the upper left side in FIG. 9). That is, in order to convert the coordinates of the measurement device 10-7 (the lower left side in FIG. 9) into the local coordinate system of the measurement device 10-1 (the upper left side in FIG. 9), the coordinates of the measurement device 10-7 (the lower left side in FIG. 9) may be multiplied by the fifth rotation matrix R₅.

In FIG. 8, in order to convert the coordinates of the measurement device 10-6 (the right side in FIG. 8) into the local coordinate system of the measurement device 10-1 (the upper side in FIG. 6), the coordinates of the measurement device 10-6 (the right side in FIG. 8) may be multiplied by the first rotation matrix R₁ and then multiplied by the fourth rotation matrix R₄. In order to convert the coordinates of the measurement device 10-8 (the left side in FIG. 8) into the local coordinate system of the measurement device 10-1 (the upper side in FIG. 6), the coordinates of the measurement device 10-8 (the left side in FIG. 8) may be multiplied by the second rotation matrix R₂ and then multiplied by the fourth rotation matrix R₄. In order to convert the coordinates of the measurement device 10-7 (the lower side in FIG. 8) into the local coordinate system of the measurement device 10-1 (the upper side in FIG. 6), the coordinates of the measurement device 10-7 (the lower side in FIG. 8) may be multiplied by the third rotation matrix R₃ and then multiplied by the fourth rotation matrix R₄.

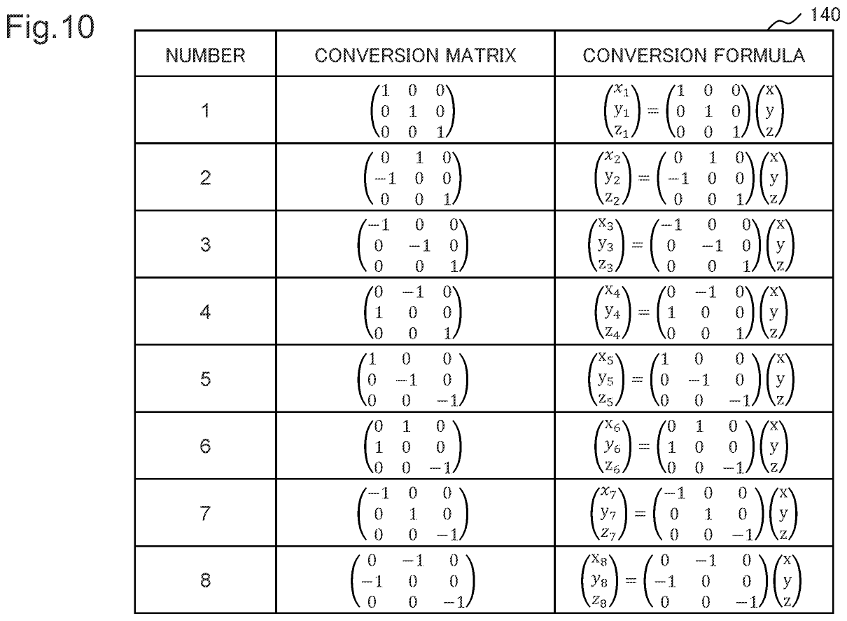

FIG. 10 is a conversion table 140 in which the conversion matrices for converting the coordinates of the measurement device 10 into the local coordinate system in the normal mounting direction (the first mounting direction) in accordance with the mounting direction of the sensor 11 are collected. The number of the conversion table 140 is a number associated with the mounting direction of the sensor 11 illustrated in FIGS. 6 to 8 (the number at the end of the measurement devices 10-1 to 8). In the conversion table 140, a conversion matrix and a conversion formula related to the mounting direction of each number are shown. A column vector on the right side of the conversion formula is the sensor data measured by the sensor 11. The left side of the conversion formula is the sensor data after the conversion into the local coordinate system in the normal mounting direction. The gait measurement unit 12 retains the conversion table 140 set in advance. The conversion table 140 is used for the conversion of the local coordinate system of the sensor data in the measurement mode.

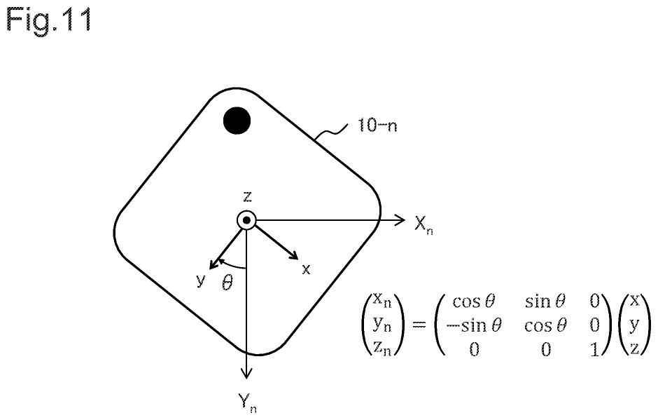

FIG. 11 is a conceptual diagram illustrating an example in which the mounting direction of a measurement device 10-n mounted at a rotation angle θ (a clockwise direction is positive) around the third axis (the z axis) with respect to the world coordinate system is converted into the normal mounting direction. FIG. 11 illustrates a conversion formula for converting the coordinates of the measurement device 10-n mounted at the rotation angle θ into the local coordinate system in the normal mounting direction (Formula 6 described below).

$$\begin{matrix}[ Formula 6 ] & \\ ( \begin{matrix}{x}_{n} \\ {y}_{n} \\ {z}_{n}\end{matrix} ) = ( \begin{matrix}cos θ & sin θ & 0 \\ - s in θ & cos θ & 0 \\ 0 & 0 & 1\end{matrix} ) ( \begin{matrix}x \\ y \\ z\end{matrix} ) & ( 6 )\end{matrix}$$

In Formula 6 described above, the sign (positive or negative) of the rotation angle θ may be set in accordance with the definition of the positive or negative of the rotation direction.

The left side of Formula 6 described above, that is, the left side of the conversion formula is the sensor data after the conversion into the local coordinate system in the normal mounting direction. By using Formula 6, the coordinates of the measurement device 10 mounted at the rotation angle θ around the third axis (the z axis) can be converted into the local coordinate system in the normal mounting direction.

When determining the mounting direction of the sensor 11, the gait measurement unit 12 shifts to the measurement mode. Similarly to the stable walking determination mode, the measurement mode is a normal-power mode in which all the spatial accelerations/spatial angular velocities are continuously measured.

In the measurement mode, the acquisition unit 121 acquires the sensor data such as an angular velocity and an acceleration measured by the acceleration sensor 111 and the angular velocity sensor 112 included in the sensor 11. The acquisition unit 121 outputs the acquired sensor data to the coordinate conversion unit 125.

The coordinate conversion unit 125 converts the local coordinate system of the sensor data acquired from the sensor 11 into a local coordinate system in a state where the sensor 11 is mounted in the normal mounting direction. In other words, the coordinate conversion unit 125 converts the coordinates of the sensor data acquired from the sensor 11 into the coordinates of the local coordinate system in a state where the sensor 11 is mounted in the normal mounting direction. For example, the coordinate conversion unit 125 converts the local coordinate system of the sensor data in accordance with the conversion table 140 registered in advance. The details of the conversion of the local coordinate system will be described later. The coordinate conversion unit 125 converts the local coordinate system converted in accordance with the mounting direction of the sensor 11 into the world coordinate system. In other words, the coordinate conversion unit 125 converts the coordinates converted in accordance with the mounting direction of the sensor 11 into coordinates in the world coordinate system. For example, the coordinate conversion unit 125 generates time-series data (the walking waveform) of the sensor data converted into the world coordinate system with regard to the acceleration or the velocity in the three axial directions, the position (the trajectory), and angular velocity and the angle around the three axes. In the present example embodiment, the walking waveform does not represent the time-series data of the sensor data as a graph, but indicates the time-series data itself of the sensor data. The coordinate conversion unit 125 stores the generated walking waveform in the storage unit 126.

The walking waveform generated by the coordinate conversion unit 125 is stored in the storage unit 126. The walking waveform stored in the storage unit 126 is used for the detection of the walking event by the detection unit 127.

The detection unit 127 acquires the walking waveform stored in the storage unit 126. The detection unit 127 detects a predetermined walking event from the walking waveform based on a feature appearing in the acquired walking waveform. The detection unit 127 outputs the detected walking event to the calculation unit 128. For example, the gait measurement unit 12 detects a characteristic change associated with the appearance of the walking event in the walking waveform. For example, the gait measurement unit 12 detects a characteristic maximum or minimum associated with the appearance of the walking event in the walking waveform.

For example, the detection unit 127 detects the central timing of the stance phase from the walking waveform of the roll angle as the predetermined walking event. When a rotation in a dorsiflexion direction is negative and a rotation in a plantarflexion direction is positive, the timing when the walking waveform of the roll angle is minimum is associated with the timing of the start of the stance phase (the heel strike) (also referred to as a stance phase start time). The timing when the walking waveform is maximum is associated with the timing of the start of the swing phase (the toe off) (also referred to as swing phase start time). The timing at the midpoint between the start of the stance phase and the start of the swing phase is associated with the central timing of the stance phase (also referred to as the mid-stance period). The detection unit 127 sets the timing of the mid-stance period to the time of the start point of one gait cycle (also referred to as a start point time). The detection unit 127 sets the timing of the mid-stance period following the start point time to the time of the end point of one gait cycle (also referred to as an end point time).

In practice, the timing when the roll angle indicates the maximum/minimum does not completely coincide with the timing of the toe off/heel strike. Therefore, the detection unit 127 may normalize the walking waveform in such a way that the timing at which the roll angle indicates maximum/minimum coincides with the timing of toe off/heel strike. For example, the detection unit 127 normalizes the walking waveform so that a section from the start point time to the swing phase start time is 30% of one gait cycle. The detection unit 127 normalizes the walking waveform so that a section from the swing phase start time to the stance phase start time is 40% of one gait cycle. The detection unit 127 normalizes the walking waveform so that a section from the stance phase start time to the end point time is 30% of one gait cycle. By normalizing the gait cycle of the walking waveform, the timings of the appearance of different walking events in accordance with a walking state and an individual difference can be made comparable.

For example, the detection unit 127 may detect the timing of the toe off/heel strike from the walking waveform of the traveling-direction acceleration (the Y-direction acceleration). In the walking waveform of the Y-direction acceleration for one gait cycle, two main peaks (a first peak and a second peak) appear. The first peak appears when the gait cycle is 20 to 40%. The first peak includes two minimum peaks and one maximum peak. The timing of the maximum peak included in the first peak is associated with the timing of the toe off. The second peak appears when the gait cycle is 50 to 70%. The second peak includes a maximum peak when the gait cycle exceeds 60% and a minimum peak when the gait cycle is 70%. The timing of the midpoint between the maximum peak and the minimum peak included in the second peak is associated with the timing of the heel strike. The minimum timing of a gentle peak between the first peak and the second peak is associated with the timing of the foot adjacent. For example, the detection unit 127 may detect, as the walking event, the tibia vertical, the foot adjacent, the heel rise, the opposite toe off, and the opposite heel strike. A method for detecting such walking events will be omitted.

The calculation unit 128 calculates a gait parameter based on the detected walking event. For example, the calculation unit 128 calculates the gait parameter using the timing of the detected walking event and the value of the sensor data at the timing of such walking events. For example, the calculation unit 128 calculates the gait parameter for each gait cycle. For example, the calculation unit 128 calculates the gait parameter such as a walking speed, a stride length, a ground-contact angle, a ground-off angle, a maximum foot rise height (a sensor position), a division (a traveling-direction trajectory), and a toe direction. The description of a method for calculating such a gait parameter will be omitted. The calculation unit 128 stores the calculated gait parameter in a buffer (not illustrated) such as an EEPROM. The buffer may be provided in a part of the storage unit 126.

The transmission unit 129 transmits the digital data stored in the buffer at a predetermined timing. For example, the transmission unit 129 transmits the gait parameter during the swing phase in which the measurement of the sensor data is hardly affected. For example, the transmission unit 129 transmits the gait parameter for each step. For example, the transmission unit 129 may transmit the gait parameter for each gait cycle. For example, the transmission unit 129 may transmit the gait parameter every 1 second. The transmission unit 129 deletes the sensor data used for the calculation of the transmitted gait parameter from the storage unit 126 (the buffer).

The gait parameter transmitted from the transmission unit 129 is received by a mobile terminal (not illustrated) carried by the user. The transmission unit 129 may transmit the gait parameter via a wire such as a cable, or may transmit the gait parameter via wireless communication. For example, the transmission unit 129 is configured to transmit the gait parameter via a wireless communication function (not illustrated) conforming to a standard such as Bluetooth (registered trademark). The communication function of the transmission unit 129 may conform to a standard other than Bluetooth (registered trademark).

The mobile terminal (not illustrated) is a communication device that can be carried by the user. For example, the mobile terminal is a portable terminal device having a communication function, such as a smart phone, a smart watch, a tablet, or a mobile phone. The mobile terminal receives the gait parameter from the measurement device 10. For example, the mobile terminal executes data processing relevant to the physical condition of the user by using the received gait parameter with application software or the like installed in the mobile terminal. For example, the mobile terminal displays a result of the data processing of the gait parameter on a screen of the mobile terminal. For example, the result of the data processing of the gait parameter may be displayed on a screen of a terminal device (not illustrated) visible by the user. For example, the mobile terminal displays any numerical value of the gait parameter received from the gait measurement unit 12 on the screen in real time. For example, the mobile terminal displays the time-series data of the gait parameter received from the gait measurement unit 12 on the screen in real time. Furthermore, the mobile terminal may transmit the received gait parameter to a server, a cloud, or the like. There is no particular limitation on an application of the gait parameter received by the mobile terminal.

### (Operation)

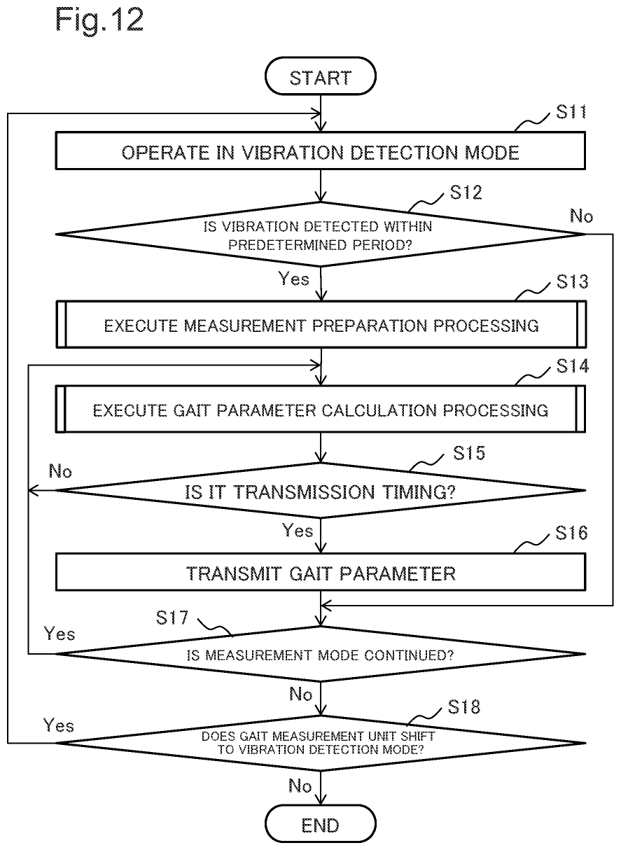

Next, an example of the operation of the measurement device 10 will be described with reference to the drawings. FIG. 12 is a flowchart for illustrating an example of the operation of the measurement device 10. In the description of processing along the flowchart in FIG. 12, the gait measurement unit 12 of the measurement device 10 is set as an operation subject.

In FIG. 12, first, the gait measurement unit 12 is operated in the vibration detection mode (step S11). For example, the gait measurement unit 12 is activated in accordance with the manipulation of the user and operated in the vibration detection mode. For example, the gait measurement unit 12 may be set to be activated in a time zone or timing set in advance.

When the vibration is detected within a predetermined period during the operation in the vibration detection mode (Yes in step S12), the gait measurement unit 12 shifts to the stable walking determination mode and executes measurement preparation processing (step S13). The gait measurement unit 12 detects a vibration derived from walking in accordance with the value of the vertical-direction acceleration (the z-direction acceleration). The measurement preparation processing is processing of determining the mounting direction of the sensor 11. The details of the measurement preparation processing will be described later. When the vibration is not detected within the predetermined period during the operation in the vibration detection mode (No in step S12), the processing proceeds to step S17.

After the measurement preparation processing in step S13, the gait measurement unit 12 executes gait parameter calculation processing (step S14). In the gait parameter calculation processing, the gait measurement unit 12 detects the walking event from the sensor data and calculates the gait parameter in accordance with the detected walking event. The details of the gait parameter calculation processing in step S14 will be described later.

When it is the timing of transmitting the gait parameter (Yes in step S15), the gait measurement unit 12 transmits the gait parameter (step S16). When it is not the timing of transmitting the gait parameter (No in step S15), the processing returns to step S14.

After step S16 or in the case of No in step S12, the gait measurement unit 12 determines the continuation of the measurement mode (step S17). When the measurement mode is continued (Yes in step S17), the processing returns to step S14. When the measurement mode is not continued (No in step S17), the processing proceeds to step S18. The continuation of the measurement mode may be determined in accordance with a condition set in advance. For example, when the predetermined period has not elapsed since the walking is detected, the measurement mode is continued. For example, when the traveling-direction acceleration exceeds a predetermined value, the measurement mode is continued.

When the measurement mode is not continued (No in step S17), the gait measurement unit 12 determines whether to shift to the vibration detection mode (step S18). When shifting to the vibration detection mode (Yes in step S18), the processing returns to step S11. When the gait measurement unit does not shift to the vibration detection mode (No in step S18), the processing according to the flowchart in FIG. 12 is ended. Whether to shift to the vibration detection mode may be determined in accordance with a predetermined timing, a stop manipulation of the user, or the like.

### [Measurement Preparation Processing]

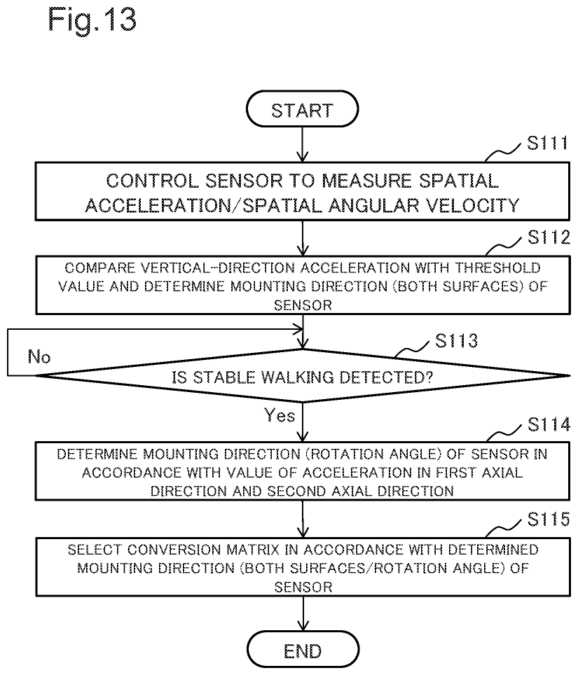

Next, an example of the measurement preparation processing (step S13 in FIG. 12) by the measurement device 10 will be described with reference to the drawings. FIG. 13 is a flowchart for illustrating an example of the measurement preparation processing by the measurement device 10. In the description of processing along the flowchart in FIG. 13, the gait measurement unit 12 of the measurement device 10 is set as an operation subject.

In FIG. 13, first, the gait measurement unit 12 shifts to the stable walking determination mode, and controls the sensor 11 to measure the spatial acceleration/spatial angular velocity (step S111).

Next, the gait measurement unit 12 compares the vertical-direction acceleration (the z-direction acceleration) with a threshold value, and determines the mounting direction (the both surfaces) of the sensor 11 (step S112). The gait measurement unit 12 determines the mounting direction (the both surfaces) of the sensor 11 by threshold determination of two systems.

Here, when the stable walking is detected (Yes in step S113), the gait measurement unit 12 determines the mounting direction (the rotation) of the sensor 11 in accordance with the value of the acceleration in the first axial direction and the second axial direction (step S114). The gait measurement unit 12 detects the stable walking when the value of acceleration in either the first axial direction or the second axial direction exceeds a threshold value. When the stable walking is not detected (No in step S113), the gait measurement unit 12 waits until the stable walking is detected. When a waiting time set in advance is exceeded, the processing proceeds to step S18 in FIG. 12.

After step S114, the gait measurement unit 12 selects a conversion matrix (a conversion formula) in accordance with the determined mounting direction (the both surfaces/rotation angle) of the sensor (step S115).

### [Gait Parameter Calculation Processing]

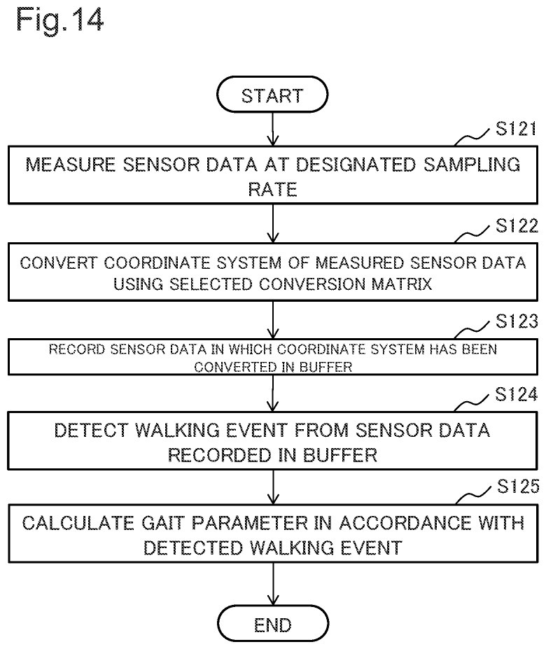

Next, an example of the gait parameter calculation processing (step S14 in FIG. 12) by the measurement device 10 will be described with reference to the drawings. FIG. 14 is a flowchart for illustrating an example of the gait parameter calculation processing by the measurement device 10. In the description of processing along the flowchart in FIG. 14, the gait measurement unit 12 of the measurement device 10 is set as an operation subject.

In FIG. 14, first, the gait measurement unit 12 measures the sensor data at a designated sampling rate (step S121). The gait measurement unit 12 acquires the sensor data including the spatial acceleration and the spatial angular velocity from the sensor 11.

Next, the gait measurement unit 12 converts the coordinate system of the measured sensor data using the selected conversion matrix (step S122). The gait measurement unit 12 converts the local coordinate system in accordance with the mounting direction of the sensor 11, and converts the converted local coordinate system into the world coordinate system. For example, the gait measurement unit 12 selects a conversion matrix according to the mounting direction of the sensor 11 with reference to the conversion table in which the conversion matrices and the conversion formulae are collected.

Next, the gait measurement unit 12 records the sensor data in which the coordinates have been converted in the buffer (the storage unit 126) (step S123).

Next, the gait measurement unit 12 detects the walking event from the sensor data recorded in the buffer (step S124).

Next, the gait measurement unit 12 calculates the gait parameter in accordance with the detected walking event (step S125). For example, the gait measurement unit 12 calculates the gait parameter such as a walking speed, a stride length, a ground-contact angle, a ground-off angle, a maximum foot rise height (a sensor position), a division (a traveling-direction trajectory), and a toe direction.

As described above, the measurement device of the present example embodiment includes the sensor and the gait measurement unit. The sensor includes the acceleration sensor that measures the acceleration in the three axial directions and the angular velocity sensor that measures the angular velocity around the three axes. The sensor outputs the sensor data measured by the acceleration sensor and the angular velocity sensor to the measurement unit.

The gait measurement unit includes the acquisition unit, the vibration detection unit, the mounting-direction-determination unit, the coordinate conversion unit, the storage unit, the detection unit, the calculation unit, and the transmission unit. In addition, the gait measurement unit includes the conversion table. The acquisition unit acquires the sensor data measured by the sensor mounted on the footwear. In the vibration detection mode, the vibration detection unit detects the start of walking in accordance with the value of the acceleration in the vertical axial direction perpendicular to the first surface of the sensor. In the stable walking determination mode, the mounting-direction-determination unit determines the mounting direction of the sensor using the acquired sensor data. In the measurement mode, the coordinate conversion unit refers to the conversion table in accordance with the determined mounting direction of the sensor, and selects the conversion formula for converting the local coordinate system of the sensor to coincide with the local coordinate system in the first mounting direction. The conversion table is a table in which the conversion formulae including the conversion matrix for converting the local coordinate system of the sensor into the local coordinate system in the first mounting direction are collected in accordance with the mounting direction of the sensor. The coordinate conversion unit converts the local coordinate system of the sensor to coincide with the local coordinate system in the first mounting direction by using the selected conversion formula. The coordinate conversion unit stores the sensor data in which the coordinate system has been converted in the storage unit. The detection unit detects the walking event from the time-series data of the sensor data stored in the storage unit. The calculation unit calculates the gait parameter in accordance with the detected walking event. The transmission unit transmits the calculated gait parameter.

The measurement device of the present example embodiment determines the mounting direction of the sensor using the sensor data. The measurement device of the present example embodiment converts the coordinate system of the sensor data in accordance with the determined mounting direction of the sensor. The measurement device of the present example embodiment calculates the gait parameter using the sensor data in which the coordinate system has been converted. Therefore, according to the measurement device of the present example embodiment, it is possible to measure the sensor data relevant to the motion of the foot regardless of the mounting direction of the sensor.

In a general measurement device, firmware optimized for the traveling direction is implemented. Therefore, in the general measurement device, various threshold determinations are changed when the mounting direction of the sensor is not normal, and thus the gait measurement is not capable of being performed unless the sensor is mounted again. For example, in a case where the firmware according to the mounting direction of the sensor is implemented, the gait can be measured by changing the firmware in accordance with the mounting direction of the sensor. However, in such a case, it is necessary to prepare the update of the firmware that is wirelessly executed from the mobile terminal for each measurement device mounted on the right and left footwear. Changing/updating the firmware for each measurement device mounted on the right and left footwear causes an increase in the management cost. In a case where the specification of the firmware for update is wrong, there is a possibility that the gait measurement is not capable of being performed in all the measurement devices mounted on the right and left footwear.

According to the present example embodiment, the coordinate system of the sensor data is converted in accordance with a determination result of the mounting direction of the sensor, and the gait parameter is calculated using the sensor data in which the coordinate system has been converted. Therefore, according to the present example embodiment, since individual firmware is not implemented on the sensor in accordance with the mounting direction, there is no factor of increasing the management cost.

In one aspect of the present example embodiment, the mounting-direction-determination unit determines that the first surface of the sensor is mounted upward when the vertical-axial acceleration in the vertical direction with respect to the first surface of the sensor exceeds the value obtained by adding the first threshold value to the gravitational acceleration. When the vertical-axial acceleration falls below the negative value of the value obtained by adding the first threshold value to the gravitational acceleration, the mounting-direction-determination unit determines that the sensor is mounted such that the first surface is directed downward. The mounting-direction-determination unit determines that the sensor is mounted such that the axial direction in which the absolute value of the acceleration indicates the maximum value is directed in the traveling direction with regard to the front-back axial direction and the right-left axial direction orthogonal to the vertical direction. The coordinate conversion unit converts the local coordinate system of the sensor in accordance with the local coordinate system in the first mounting direction in accordance with the determined mounting direction of the sensor. According to the present aspect, the mounting direction (the both surfaces) of the sensor is determined in accordance with the value of the vertical-axial acceleration, and the mounting direction (the rotation) of the sensor is determined in accordance with the value of the acceleration in the front-back axial direction and the right-left axial direction, whereby the mounting direction of the sensor can be determined.

In one aspect of the present example embodiment, the mounting-direction-determination unit determines that the sensor is mounted such that the first surface is directed upward when the vertical-direction acceleration in the vertical direction with respect to the first surface of the sensor exceeds the value obtained by adding the first threshold value to the gravitational acceleration. The mounting-direction-determination unit determines that the sensor is mounted such that the first surface is directed downward when the vertical-direction acceleration falls below the negative value of the value obtained by adding the first threshold value to the gravitational acceleration. The mounting-direction-determination unit determines the traveling direction in accordance with the ratio of the maximum value of the absolute value of the acceleration with regard to the front-back axial direction and the right-left axial direction orthogonal to the vertical direction. The coordinate conversion unit converts the local coordinate system of the sensor in accordance with the local coordinate system in the first mounting direction in accordance with the determined mounting direction of the sensor. According to the present aspect, the mounting direction (the both surfaces) of the sensor is determined in accordance with the value of the vertical-axial acceleration, and determines the mounting direction (the rotation) of the sensor in accordance with the ratio of the maximum value of the absolute value of the acceleration in the front-back axial direction and the right-left axial direction, whereby the mounting direction of the sensor can be determined.

In one aspect of the present example embodiment, the coordinate conversion unit converts the local coordinate system of the sensor to coincide with the local coordinate system in the first mounting direction by using the conversion formula according to the determined mounting direction of the sensor. The conversion formula includes the conversion matrix for converting the local coordinate system of the sensor into the local coordinate system in the first mounting direction. According to the present aspect, the local coordinate system of the sensor can be converted into the local coordinate system in the first mounting direction by using the conversion formula for each mounting direction.

In one aspect of the present example embodiment, the coordinate conversion unit converts the local coordinate system of the sensor to coincide with the local coordinate system in the first mounting direction by using the conversion formula according to the determined rotation angle in the mounting direction of the sensor in the front-back axial direction and the right-left axial direction. The conversion formula includes the rotation matrix for converting the local coordinate system of the sensor into the local coordinate system in the first mounting direction. According to the present aspect, the local coordinate system of the sensor can be converted into the local coordinate system in the first mounting direction by using the conversion formula for each mounting direction. The local coordinate system of the sensor can be converted into the local coordinate system in the first mounting direction by using the conversion formula according to the rotation angle in the mounting direction in the front-back axial direction and the right-left axial direction.

### Second Example Embodiment

Next, a gait measurement system according to a second example embodiment will be described with reference to the drawings. The gait measurement system of the present example embodiment includes the measurement device of the first example embodiment. The gait measurement system of the present example embodiment executes the data processing relevant to the physical condition of the user by using the gait parameter output from the measurement device.

### (Configuration)

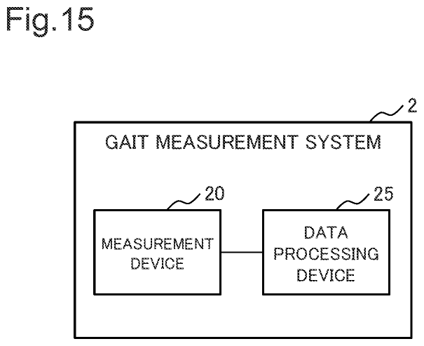

FIG. 15 is a block diagram illustrating an example of the configuration of a gait measurement system 2 according to the present example embodiment. The gait measurement system 2 includes a measurement device 20 and a data processing device 25.

The measurement device 20 has the same configuration as the measurement device 10 of the first example embodiment. The measurement device 20 is installed on the footwear of the user. When detecting the vibration during the operation in the vibration detection mode, the measurement device 20 shifts to the stable walking determination mode. When shifting to the stable walking determination mode, the measurement device 20 determines the mounting direction of the own device (the measurement device 20). When the mounting direction is determined, the measurement device 20 shifts to the measurement mode. In the measurement mode, the measurement device 20 acquires the sensor data such as an angular velocity and an acceleration. The measurement device 20 converts the coordinate system of the acquired sensor data in accordance with the determined mounting direction. The measurement device 20 detects the walking event from the time-series data of the sensor data in which the coordinate system has been converted. The measurement device 20 calculates the gait parameter in accordance with the detected walking event. The measurement device 20 transmits the calculated gait parameter to the data processing device 25.

For example, the measurement device 20 transmits the gait parameter at the timing of the swing phase. For example, the measurement device 20 transmits the gait parameter for each step. For example, the measurement device 20 may transmit the gait parameter for each gait cycle. The measurement device 20 deletes the sensor data used for the calculation of the transmitted gait parameter from the buffer.

The gait parameter transmitted from the measurement device 20 is received by a mobile terminal (not illustrated) carried by the user. The measurement device 20 may transmit the gait parameter via a wire such as a cable or may transmit the gait parameter via wireless communication. For example, the measurement device 20 is configured to transmit the gait parameter via a wireless communication function (not illustrated) conforming to a standard such as Bluetooth (registered trademark). The communication function of the measurement device 20 may conform to a standard other than Bluetooth (registered trademark).

The mobile terminal (not illustrated) is a communication device that can be carried by the user. For example, the mobile terminal is a communication device having a communication function, such as a smart phone, a smart watch, or a mobile phone. The mobile terminal receives the gait parameter from the measurement device 20. For example, the mobile terminal processes the received gait parameter by the data processing device 25 installed in the mobile terminal. For example, the mobile terminal transmits the received gait parameter to the data processing device 25 implemented in a server (not illustrated) or a cloud (not illustrated). In the present example embodiment, it is assumed that the data processing device 25 is installed in a mobile terminal. The data processing device 25 may be a device specialized in the data processing of the gait parameter from the measurement device 20.

The data processing device 25 acquires the gait parameter from the measurement device 20. The data processing device 25 executes the data processing relevant to the physical condition according to the gait of the user by using the gait parameter acquired from the measurement device 20.

For example, the data processing device 25 determines the symmetry of the walking of the user by using the gait parameter. For example, the data processing device 25 estimates the degree of progression of the hallux valgus of the user using the gait parameter. For example, the data processing device 25 performs personal identification of the user or personal authentication of the user by using the gait parameter. For example, the data processing device 25 calculates the step length and the stride length of the user by using the gait parameter. For example, the data processing device 25 estimates the degree of pronation/supination of the user by using the gait parameter. For example, the data processing device 25 performs measurement relevant to the lower limb of the user by using the gait parameter. The data processing by the data processing device 25 is not limited to the example described herein as long as the gait parameter acquired from the measurement device 20 is used. A specific method of the data processing by the data processing device 25 will not be described.

The data processing device 25 outputs a result of the data processing of the gait parameter. For example, the data processing device 25 displays the result of the data processing of the gait parameter on a screen of a mobile terminal in which the data processing device 25 is installed. For example, the data processing device 25 displays any numerical value of the gait parameter received from the measurement device 20 on the screen of the mobile terminal in real time. For example, the data processing device 25 displays the time-series data of the gait parameter received from the measurement device 20 on the screen of the mobile terminal in real time. For example, the data processing device 25 displays information relevant to the physical condition of the user estimated by using the gait parameter received from the measurement device 20 and information according to the estimated physical condition on the screen of the mobile terminal. For example, the data processing device 25 may transmit the received gait parameter to a server, a cloud, or the like. There is no particular limitation on an application of the gait parameter received by the mobile terminal.

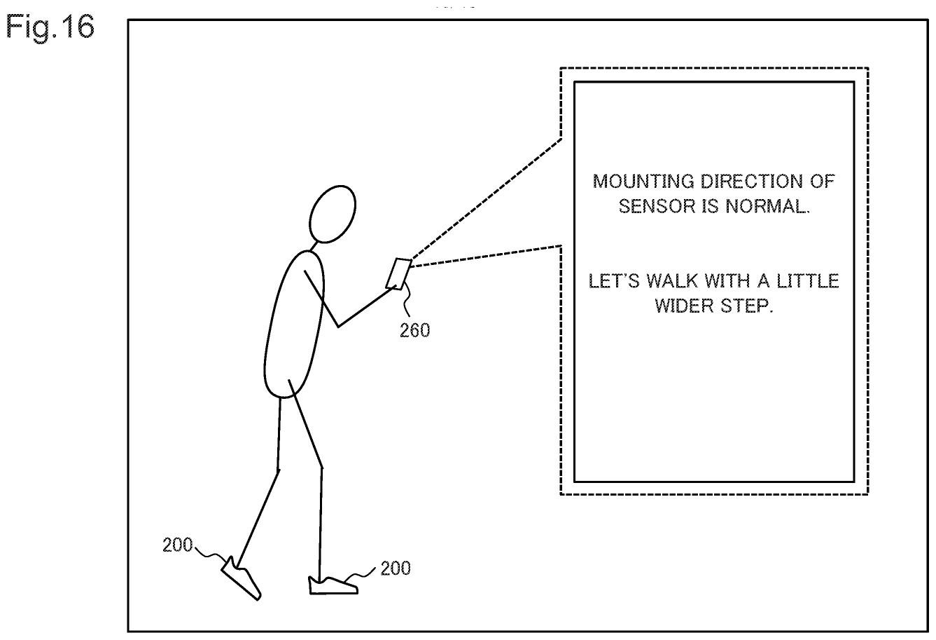

FIG. 16 illustrates an example in which information according to the walking of the user is displayed on a screen of a mobile terminal 260 carried by the user walking in shoes 200 on which the measurement device 20 is installed. In the example of FIG. 16, recommendation information according to the physical condition of the user estimated by using the gait parameter received from the measurement device 20 is displayed on the screen of the mobile terminal 260. In the example of FIG. 16, information according to the mounting direction of the sensor 11, such as “MOUNTING DIRECTION OF SENSOR IS NORMAL.” is displayed on the screen of the mobile terminal 260 in accordance with the determined mounting direction of the sensor. In the example of FIG. 16, recommendation information such as “LET'S WALK WITH A LITTLE WIDER STEP.” is displayed on the screen of the mobile terminal 260 in accordance with the physical condition of the user estimated by using the gait parameter (the stride). The user who has confirmed the recommendation information displayed on the screen of the mobile terminal 260 may be able to improve the own health condition by improving the walking in accordance with the recommendation information.

For example, the data processing device 25 estimates a foot symptom and a recovery degree from an injury in accordance with a variation in the right and left strides. For example, in a case where the variation in the right and left strides increases, there is a possibility that the symptom progresses or the injury worsens. In such a case, there is a possibility that the symptom and the injury of the user can be improved by displaying the information for recommending a medical examination in the hospital on the screen of the mobile terminal 260 of the user. For example, in a case where the variation in the right and left strides decreases, there is a possibility that the user tends to recover from the symptom or the injury. In such a case, in a case where information indicating that the user tends to recover from the symptom or the injury is displayed on the screen of the mobile terminal 260 of the user, there is a possibility that the motivation of the user such as rehabilitation is improved.

For example, in a case where the motion of the ankle is affected by a sprain or an old wound of the foot, such an influence is reflected on the value of the ground-contact angle/ground-off angle and a right/left balance. Therefore, it is possible to verify the degree and state of recovery of the sprain or the old wound in accordance with the magnitude of the value of the ground-contact angle/ground-off angle and the right/left balance. For example, in a case where the value of the ground-contact angle/ground-off angle of the foot having the sprain or the old wound falls below a predetermined value, there is a possibility that the symptom of the user can be improved by displaying information for recommending an examination or a treatment on the screen of the mobile terminal 260 of the user. For example, in a case where the value of the ground-contact angle/ground-off angle of the foot having the sprain or the old wound exceeds the predetermined value, there is a possibility that the life quality of the user is improved by displaying the information indicating that the user tends to recover from the symptom or the injury on the screen of the mobile terminal 260 of the user.

For example, when the foot rise height associated with the absolute value of the clearance decreases, a risk of falling over a step or the like increases. Therefore, the falling risk can be verified by verifying the foot rise height. For example, in a case where the foot rise height falls below a predetermined value, there is a possibility that the falling risk of the user can be avoided by displaying information for recommending an examination, a treatment, or training on the screen of the mobile terminal 260 of the user. For example, in a case where the foot rise height exceeds the predetermined value, there is a possibility that the life quality of the user is improved by displaying information indicating that the user is in a healthy walking state on the screen of the mobile terminal 260 of the user.

For example, in a situation of visiting the hospital for the rehabilitation of the symptom or the injury of the foot, the user walks in front of a doctor, and the doctor determines the state of the foot. However, in front of the doctor, there may be a case where walking different from the daily walking is exhibited depending on the psychological state of the user. Therefore, it is desirable that the physical condition can be determined based on numerical values and indices measured in daily life. Since the gait measurement system of the present example embodiment is capable of measuring/estimating the numerical value and the index indicating the state of the foot in daily life, it is easy to obtain accurate determination without being affected by the psychological state of the user. Since the gait measurement system of the present example embodiment is capable of grasping the state of the user in real time in daily life, even in a case where a symptom or a medical condition rapidly deteriorates, the gait measurement system is capable of responding flexibly by making emergency contact with the hospital or the like.

As described above, the gait measurement system of the present example embodiment includes the measurement device and the data processing device. The measurement device includes the sensor and the gait measurement unit. The sensor includes the acceleration sensor that measures the acceleration in the three axial directions and the angular velocity sensor that measures the angular velocity around the three axes. The gait measurement unit converts the coordinate system of the sensor data measured by the acceleration sensor and the angular velocity sensor in accordance with the mounting direction of the sensor. The gait measurement unit calculates the gait parameter by using the sensor data in which the coordinate system has been converted. The gait measurement unit transmits the calculated gait parameter to the data processing device. The data processing device acquires the gait parameter transmitted by the measurement device installed in the foot portion of the user. The data processing device executes the data processing relevant to the physical condition of the user by using the gait parameter. For example, the data processing device displays the information relevant to the physical condition of the user obtained by the data processing using the gait parameter on the screen of the terminal device visible by the user.

The gait measurement system of the present example embodiment calculates the gait parameter by using the sensor data in which the coordinate system has been converted in accordance with the mounting direction of the sensor. Therefore, according to the gait measurement system of the present example embodiment, it is possible to measure the sensor data relevant to the motion of the foot regardless of the mounting direction of the sensor. According to the gait measurement system of the present example embodiment, the user oneself can confirm the physical condition of the user displayed on the screen of the terminal device.

### Third Example Embodiment

Next, a gait measurement device according to a third example embodiment will be described with reference to the drawings. The gait measurement device of the present example embodiment has a configuration in which the measurement unit of the first example embodiment is simplified.

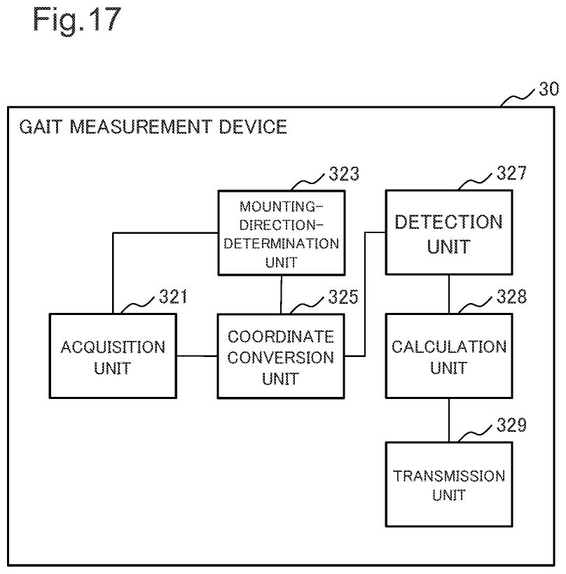

FIG. 17 is a block diagram illustrating an example of the configuration of a gait measurement device 30 according to the present example embodiment. The gait measurement device 30 includes an acquisition unit 321, a mounting-direction-determination unit 323, a coordinate conversion unit 325, a detection unit 327, a calculation unit 328, and a transmission unit 329.

The acquisition unit 321 acquires sensor data measured by a sensor mounted on the footwear. The mounting-direction-determination unit 323 determines the mounting direction of the sensor using the acquired sensor data. The coordinate conversion unit 325 converts the coordinate system of the sensor data in accordance with the determined mounting direction of the sensor. The detection unit 327 detects a walking event from the time-series data of the sensor data in which the coordinate system has been converted. The calculation unit 328 calculates a gait parameter in accordance with the detected walking event. The transmission unit 329 transmits the calculated gait parameter.

The gait measurement device of the present example embodiment determines the mounting direction of the sensor using the sensor data, and converts the coordinate system of the sensor data in accordance with the determined mounting direction of the sensor. The gait measurement device of the present example embodiment calculates the gait parameter using the sensor data in which the coordinate system has been converted. Therefore, according to the gait measurement device of the present example embodiment, the sensor data relevant to the motion of the foot can be measured regardless of the mounting direction of the sensor.

### (Hardware)

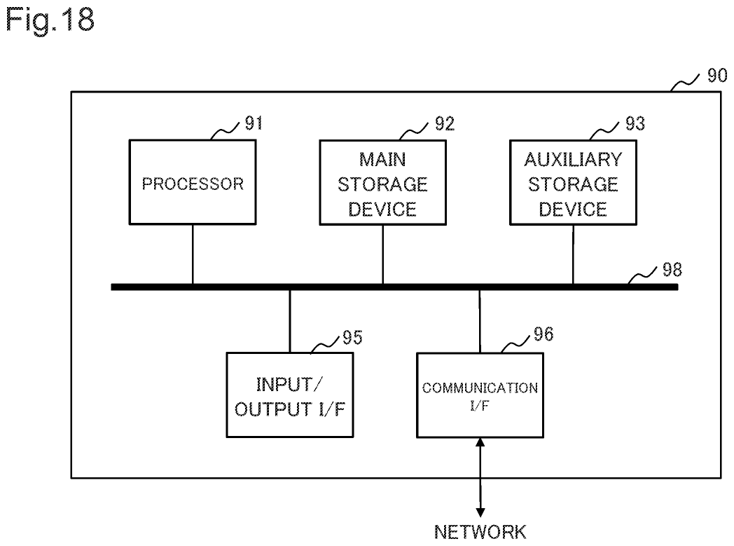

Here, a hardware configuration for executing control and processing according to each of the example embodiments of the present disclosure will be described using an information processing device 90 in FIG. 18 as an example. The information processing device 90 in FIG. 18 is a configuration example of executing the control and the processing of each of the example embodiments, and does not limit the scope of the present disclosure.

As illustrated in FIG. 18, the information processing device 90 includes a processor 91, a main storage device 92, an auxiliary storage device 93, an input/output interface 95, and a communication interface 96. In FIG. 18, the interface is abbreviated as an I/F (interface). The processor 91, the main storage device 92, the auxiliary storage device 93, the input/output interface 95, and the communication interface 96 are connected to each other via a bus 98 such that data communication is available. The processor 91, the main storage device 92, the auxiliary storage device 93, and the input/output interface 95 are connected to a network such as the Internet or an intranet via the communication interface 96.

The processor 91 decompresses a program stored in the auxiliary storage device 93 or the like in the main storage device 92. The processor 91 executes the program decompressed in the main storage device 92. In the present example embodiment, a software program installed in the information processing device 90 may be used. The processor 91 executes the control and the processing according to each of the example embodiments.

The main storage device 92 has an area in which the program is decompressed. The program stored in the auxiliary storage device 93 or the like is decompressed in the main storage device 92 by the processor 91. The main storage device 92 is enabled by, for example, a volatile memory such as a dynamic random access memory (DRAM). A nonvolatile memory such as a magnetoresistive random access memory (MRAM) may be configured/added as the main storage device 92.

The auxiliary storage device 93 stores various data pieces such as a program. The auxiliary storage device 93 is enabled by a local disk such as a hard disk or a flash memory. Various data pieces may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to an external system or device via a network such as the Internet or an intranet based on a standard or a specification. The input/output interface 95 and the communication interface 96 may be shared as an interface connected to the external device.

An input device such as a keyboard, a mouse, and a touch panel may be connected to the information processing device 90 as necessary. These input device is used to input information and setting. When the touch panel is used as the input device, the display screen of the display device may also serve as the interface of the input device. The data communication between the processor 91 and the input device may be mediated by the input/output interface 95.

The information processing device 90 may be provided with a display device for displaying information. In a case where the display device is provided, the information processing device 90 preferably includes a display control device (not illustrated) for controlling the display of the display device. The display device may be connected to the information processing device 90 via the input/output interface 95.

The information processing device 90 may be provided with a drive device. The drive device mediates the reading of data and a program from a recording medium, the writing of a processing result of the information processing device 90 to the recording medium, and the like between the processor 91 and the recording medium (a program recording medium). The drive device may be connected to the information processing device 90 via the input/output interface 95.

The above is an example of a hardware configuration for enabling the control and the processing according to each of the example embodiments of the present invention. The hardware configuration in FIG. 18 is an example of a hardware configuration for executing the control and the processing according to each of the example embodiments, and does not limit the scope of the present invention. A program for allowing a computer to execute the control and the processing according to each of the example embodiments is also included in the scope of the present invention. A program recording medium in which the program according to each of the example embodiments is recorded is also included in the scope of the present invention. The recording medium can be enabled by, for example, an optical recording medium such as a compact disc (CD) or a digital versatile disc (DVD). The recording medium may be enabled by a semiconductor recording medium such as a universal serial bus (USB) memory or a secure digital (SD) card. The recording medium may be enabled by a magnetic recording medium such as a flexible disk, or another recording medium. When the program executed by the processor is recorded in the recording medium, the recording medium is associated with the program recording medium.

The constituents of each of the example embodiments may be randomly combined. The constituents of each of the example embodiments may be enabled by software or may be enabled by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

2 gait measurement system
10, 20 measurement device
11 sensor
12 gait measurement unit
25 data processing device
30 gait measurement device
111 acceleration sensor
112 angular velocity sensor
121, 321 acquisition unit
122 vibration detection unit
123, 323 mounting-direction-determination unit
125, 325 coordinate conversion unit
126 storage unit
127, 327 detection unit
128, 328 calculation unit
129, 329 transmission unit

What is claimed is:

1. A gait measurement device, comprising:
a memory storing instructions; and
a processor connected to the memory and configured to execute the instructions to:
acquire sensor data measured by a sensor mounted on footwear;
determine a mounting direction of the sensor using the acquired sensor data;
convert a coordinate system of the sensor data in accordance with the determined mounting direction of the sensor;
detect a walking event from time-series data of the sensor data in which the coordinate system has been converted;
calculate a gait parameter in accordance with the detected walking event; and
transmit the calculated gait parameter.

2. The gait measurement device according to claim 1, wherein
the processor is configured to execute the instructions to
determine that a first surface of the sensor is mounted upward when a vertical-axial acceleration in a vertical direction with respect to the first surface of the sensor exceeds a value obtained by adding a first threshold value to a gravitational acceleration,
determine that the first surface of the sensor is mounted downward when the vertical-axial acceleration falls below a negative value of the value obtained by adding the first threshold value to the gravitational acceleration,
determine that the sensor is mounted such that an axial direction in which an absolute value of an acceleration indicates a maximum value is directed in a traveling direction with regard to a front-back axial direction and a right-left axial direction orthogonal to the vertical direction, and
convert a local coordinate system of the sensor to coincide with a local coordinate system in a first mounting direction in accordance with the determined mounting direction of the sensor.

3. The gait measurement device according to claim 1, wherein
the processor is configured to execute the instructions to
determine that a first surface of the sensor is mounted upward when a vertical-direction acceleration in a vertical direction with respect to the first surface of the sensor exceeds a value obtained by adding a first threshold value to a gravitational acceleration,
determine that the first surface of the sensor is mounted downward when the vertical-direction acceleration falls below a negative value of the value obtained by adding the first threshold value to the gravitational acceleration,
determine a traveling direction in accordance with a ratio of a maximum value of an absolute value of an acceleration with regard to a front-back axial direction and a right-left axial direction orthogonal to the vertical direction, and
convert a local coordinate system of the sensor to coincide with a local coordinate system in a first mounting direction in accordance with the determined mounting direction of the sensor.

4. The gait measurement device according to claim 2, wherein
the processor is configured to execute the instructions to
convert the local coordinate system of the sensor to coincide with the local coordinate system in the first mounting direction by using a conversion formula including a conversion matrix for converting the local coordinate system of the sensor into the local coordinate system in the first mounting direction in accordance with the determined mounting direction of the sensor.

5. The gait measurement device according to claim 2, wherein
the processor is configured to execute the instructions to
select a conversion formula that converts the local coordinate system of the sensor to coincide with the local coordinate system in the first mounting direction with reference to a conversion table in which the conversion formulae including the conversion matrix for converting the local coordinate system of the sensor into the local coordinate system in the first mounting direction in accordance with the determined mounting direction of the sensor are collected, and
convert the local coordinate system of the sensor to coincide with the local coordinate system in the first mounting direction by using the selected conversion formula.

6. The gait measurement device according to claim 2, wherein
the processor is configured to execute the instructions to
convert the local coordinate system of the sensor to coincide with the local coordinate system in the first mounting direction by using a conversion formula including a rotation matrix for converting the local coordinate system of the sensor into the local coordinate system in the first mounting direction in accordance with a determined rotation angle in the mounting direction of the sensor in the front-back axial direction and the right-left axial direction.

7. A measurement device, comprising:
the gait measurement device according to claim 1; and
a sensor that includes an acceleration sensor measuring an acceleration in three axial directions and an angular velocity sensor measuring an angular velocity around three axes, and outputs sensor data measured by the acceleration sensor and the angular velocity sensor to the gait measurement device.

8. A gait measurement system, comprising:
the measurement device according to claim 7; and
a data processing device that comprises
a memory storing instructions; and
a processor connected to the memory and configured to execute the instructions to
acquire the gait parameter transmitted by the gait measurement device installed in a foot portion of a user, and execute data processing relevant to a physical condition of the user by using the gait parameter, and
display information relevant to the physical condition of the user obtained by the data processing using the gait parameter on a screen of a terminal device visually recognizable by the user.

9. A gait measurement method for allowing a computer to:
acquire sensor data measured by a sensor mounted on footwear;
determine a mounting direction of the sensor using the acquired sensor data;
convert a coordinate system of the sensor data in accordance with the determined mounting direction of the sensor;
detect a walking event from time-series data of the sensor data in which a coordinate system has been converted;
calculate a gait parameter in accordance with the detected walking event; and
transmit the calculated gait parameter.

10. A non-transitory recording medium recording a program for allowing a computer to execute:
processing of acquiring sensor data measured by a sensor mounted on footwear;
processing of determining a mounting direction of the sensor using the acquired sensor data;
processing of converting a coordinate system of the sensor data in accordance with the determined mounting direction of the sensor;
processing of detecting a walking event from time-series data of the sensor data in which a coordinate system has been converted;
processing of calculating a gait parameter in accordance with the detected walking event; and
processing of transmitting the calculated gait parameter.
